(19) United States
(12) Patent Application Publication
(10) Pub. No.: US 2025/0292883 A1
(43) Pub. Date: Sep. 18, 2025

(54) RECURRING REMOTE MONITORING WITH REAL-TIME EXCHANGE TO ANALYZE HEALTH DATA AND GENERATE ACTION PLANS

(71) Applicant: Evernorth Strategic Development, Inc., St. Louis, MO (US)

(72) Inventors: Neha Jain, Bridgewater, NJ (US); Kevin Herzig, Fort Myers, FL (US)

(21) Appl. No.: 19/219,162

(22) Filed: May 27, 2025

Related U.S. Application Data
(63) Continuation of application No. 18/204,716, filed on Jun. 1, 2023, now Pat. No. 12,315,604.
(60) Provisional application No. 63/348,398, filed on Jun. 2, 2022.

(51) Int. Cl. G16H 10/60; G16H 20/00; G16H 40/67

(52) U.S. Cl. CPC G16H 10/60; G16H 20/00; G16H 40/67

(57) ABSTRACT

Methods, apparatuses and systems provide technology to select a given group of monitoring information to collect from a user, and instruct a first computing device associated with the user to collect the given group of monitoring information. The technology determines that the given group of monitoring information is unavailable to be provided by the first computing device, and in response to the given group of monitoring information being determined as being unavailable to be provided by the first computing device, generates an outreach event to request the given group of monitoring information from the user.

### CROSS-REFERENCE TO RELATED APPLICATIONS

The present application is a continuation of and claims the benefit of priority to U.S. patent application Ser. No. 18/204,716 (filed on Jun. 1, 2023) and U.S. Provisional Patent Application Ser. No. 63/348,398, filed Jun. 2, 2022 which are hereby incorporated by reference in their entireties.

### TECHNICAL FIELD

Embodiments generally relate to a computing platform that analyzes health data of a patient, determines if the health data indicates a health condition, and follows an action plan to mitigate and/or otherwise address the health condition. In particular, the computing platform remotely monitors the health data of the patient and provides the action plan.

### BACKGROUND

Health can be monitored remotely in a variety of ways. Certain connected devices, such as blood pressure monitors, can be used by a patient to collect health information. Such information can be provided to a communication device of the patient and transmitted to one or more other parties, such as medical providers.

### BRIEF DESCRIPTION OF THE DRAWINGS

FIG. 1 is a block diagram of an example real-time health monitoring platform, according to some embodiments.

FIG. 2 is an example database that may be deployed within the system of FIG. 1, according to some embodiments.

FIG. 3 is a block diagram of an example outreach event selection system that may be deployed within the system of FIG. 1, according to some embodiments.

FIGS. 4 and 5 are example user interfaces of the real-time health monitoring platform, according to example embodiments.

FIG. 6 is a flowchart illustrating example operations of the real-time health monitoring platform, according to example embodiments.

FIG. 7 is a block diagram illustrating an example software architecture, which may be used in conjunction with various hardware architectures herein described.

FIG. 8 is a block diagram illustrating components of a machine, according to some example embodiments.

FIG. 9 is a functional block diagram of an example neural network that can be used for the inference engine or other functions (e.g., engines) as described herein to produce a predictive model.

FIG. 10A is a diagram of an example of a health monitoring, identification, and treatment process according to an embodiment.

FIG. 10B is a diagram of an example of a health condition monitoring table according to an embodiment.

FIG. 11 is a diagram of an example of a noise reduction process according to an embodiment.

FIG. 12 is a diagram of an example of a health plan compliance process according to an embodiment.

FIG. 13 is a flowchart of an example of a method of generating an action plan according to an embodiment.

FIG. 14 is a flowchart of an example of a method of improving compliance with an action plan according to an embodiment.

FIG. 15 is a flowchart of an example of a method of switching action plans according to an embodiment.

FIG. 16 is a flowchart of an example of a method of selecting action pathways according to an embodiment.

FIG. 17 is a block diagram of an example of a computing system according to an embodiment.

FIG. 18A generally illustrates a functional block diagram of a system including a high-volume pharmacy according to the principles of the present disclosure.

FIG. 18B generally illustrates a computing device according to the principles of the present disclosure.

FIG. 19 generally illustrates a functional block diagram of a pharmacy fulfillment device, which may be deployed within the system of FIG. 18A.

FIG. 20 generally illustrates a functional block diagram of an order processing device, which may be deployed within the system of FIG. 18A.

FIG. 21 generally illustrates a personalized healthcare experience method according to the principles of the present disclosure.

FIG. 22 generally illustrates a personalized experience interface according to the principles of the present disclosure.

FIGS. 23A and 23B are graphical representations of example recurrent neural networks for generating personalized healthcare.

FIG. 24 is a graphical representation of layers of an example long short-term memory (LSTM) machine learning model.

FIG. 25 is a flowchart illustrating an example process for training a machine learning model.

FIG. 26 is a block diagram of an example personalized experience interface selection system that may be deployed within the systems described herein, according to some examples.

### DETAILED DESCRIPTION

Example methods and systems for a real-time health monitoring platform are provided. In the following description, for purposes of explanation, numerous specific details are set forth in order to provide a thorough understanding of example embodiments. It will be evident, however, to one of ordinary skill in the art that embodiments of the invention may be practiced without these specific details.

### Reoccurring Remote Monitoring Examples

Patient health information can be monitored by health providers remotely. For example, a health monitoring device, such as a blood pressure monitor, can be provided to a patient. The health monitoring device can collect a health measurement from the patient and can report back the collected health measurement to the health provider. Many times, the health information needs to be measured over a period of days, weeks or months in order to detect certain medical conditions. To do so, a patient needs to remember to use the health monitoring device to recollect the health measurement and to provide such a measurement to the provider. Having to remember to do so is incredibly inefficient and usually results in measurements being missed or skipped altogether. Some more sophisticated patients can setup manual reminders to collect the health measurements. But such reminders can be difficult to setup to be triggered at the right time and place which puts an enormous burden on patients and healthcare professionals. The healthcare professionals also need to continuously check various patients to ensure the measurements are diligently and timely collected which takes a great deal of time and resources away from performing other tasks, and is prone to error (e.g., human oversight).

Even when the measurements are collected at the right time by the patients, sometimes such measurements are not accurate. In such cases, the patient may need to re-collect the measurement after some period of time when the healthcare provider evaluates the measurement and informs the patient that the measurement was not accurate. This process is very frustrating and can cause patients to avoid interacting with the systems to remotely monitor their health.

The disclosed embodiments provide systems and methods to monitor patient health in real-time using mobile monitoring devices or equipment, such as Internet of Things (IoT) devices. The disclosed techniques select a given type (e.g., group such as blood pressure, glucose, etc.) of health monitoring information (e.g., prescription information collected by order processing device 114 and/or pharmacy fulfillment device 1812 or other device indicating that a prescription was successfully filled and collected) to collect from a patient. The disclosed techniques instruct a mobile device (e.g., a first computing device such as an IoT device) associated with the patient to collect the given type of health monitoring information. The IoT device includes at least one of a smart scale, a blood pressure cuff, a smart watch, an exercise bracelet, glucometer, a physiological sensor, and/or an ECG device. The given type of health monitoring information includes at least one of blood pressure information, aerobic activity information, body mass index (BMI) information, sodium intake information, arrhythmia information, and/or glucose information.

The disclosed techniques determine that the mobile device has failed to collect the given type of health monitoring information and, in response, generate an outreach event to request the given type of health monitoring information from the patient. The outreach event that is selected can be of a particular type that is suitable for the class of mobile device used to collect the given type of health monitoring information and/or the demographic or type of patient being monitored. This provides a very unique and tailored experience to patients in collecting their health monitoring information which improves the overall efficiency and process of collecting such information. The outreach event, in an example embodiment, can be positive reinforcement to the patient through an electronic device, a warning and/or an incentive to encourage collection of the health information from the monitoring device. The outreach event can be individualized (e.g., with a machine learning model) based on historical data about the type of patient, the medical state, the disease state, and/or a care pathway assigned to the patient.

In this way, the disclosed embodiments improve the overall process of providing medical care and specifically improves the efficiency at which health information is received from a patient. As a result, a great deal of time is saved and the patient can avoid navigating through a multitude of pages of information to provide health information. This saves time and reduces the amount of resources needed to accomplish a task. In an example, the collection of information is integrated into an interactive application run on a patient associated device. The application can be part of a care pathway (e.g., escalation, intervention, action, stable, etc.) that includes data collection events that pull sensed health data from patient assigned devices.

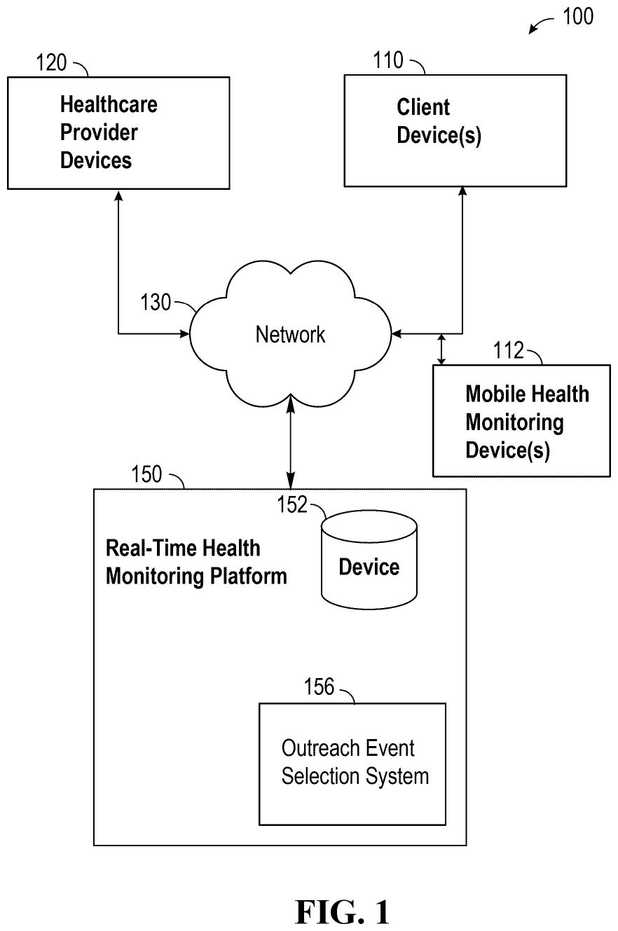

FIG. 1 is a block diagram showing an example health monitoring system 100 according to various exemplary embodiments. The health monitoring system 100 includes one or more client devices 110, one or more healthcare provider devices 120, and a real-time health monitoring platform 150, and one or more mobile health monitoring device(s) 112 that are communicatively coupled over a network 130 (e.g., Internet, telephony network). Each of the one or more mobile health monitoring device(s) 112 includes hardware and/or software configured to collect and/or measure a particular type of health monitoring information.

Each of the one or more mobile health monitoring device(s) 112 includes communication circuitry for electronically and automatically transmitting health information that is measured in real-time to the client device(s) 110, one or more healthcare provider devices 120 and/or the real-time health monitoring platform 150. For example, the one or more mobile health monitoring device(s) 112 can include IoT devices, such as a glucose monitor, a heart-rate monitor, a hand hygiene monitor, a mood monitor, a connected inhaler, a connected contact lens, a smart scale, a blood pressure cuff, a smart watch, an exercise bracelet, glucometer, an EKG device and/or an ECG device. In some cases, the mobile health monitoring device(s) 112 include storage circuitry for storing historically collected samples of the health information, such as all of the health information collected over a threshold period of time or in the lifetime of the given device. In some cases, some or all of the historical information can be stored at least partially on the client device 110 associated with the mobile health monitoring device(s) 112.

As used herein, the term “client device” may refer to any machine that interfaces to a communications network (such as network 130) to access the real-time health monitoring platform 150. The client device 110 may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network or the real-time health monitoring platform 150.

The client device 110 can establish initially a secure connection with one or more mobile health monitoring device(s) 112. After establishing the secure connection, the mobile health monitoring device(s) 112 can be configured to exchange encrypted communications in real-time with the client device 110 with which the connection was established and/or one or more dedicated healthcare provider devices 120. In this way, a patient's health monitoring information remains securely stored on the patient's devices and cannot be compromised. This ensures that the health monitoring information is maintained private. In some cases, functionality of one or more of the mobile health monitoring device(s) 112 can be implemented by the client device 110. This can avoid the need to use a separate hardware device to collect a given type of health monitoring information.

In some cases, the real-time health monitoring platform 150 is accessible over a global communication system, e.g., the Internet or world-wide web. In such instances, the real-time health monitoring platform 150 hosts a website that is accessible to the client devices 110 and/or mobile health monitoring device(s) 112. Upon accessing the website, the client devices 110 provide secure login credentials, which are used to access a profile associated with the login credentials. One or more user interfaces associated with the real-time health monitoring platform 150 are provided over the Internet via the website to the client devices 110.

Healthcare provider devices 120 can include the same or similar functionality as client devices 110 for accessing the real-time health monitoring platform 150. In some cases, the healthcare provider devices 120 are used by “internal” users. Internal users are personnel, such as physicians, clinicians, healthcare providers, health-related coaches pharmacy benefit manager (PBM) operators, pharmacists, specialty pharmacy operators or pharmacists, or the like that are associated with or employed by an organization that provides the real-time health monitoring platform 150. In some cases, the healthcare provider devices 120 are used by “external” users. External users are personnel, such as physicians, clinicians, and health-related coaches that are associated with or employed by a different (external) organization than that which provides the real-time health monitoring platform 150. In some cases, the healthcare provider devices 120 receive real-time exchanges of data, such as messages from mobile health monitoring device(s) 112, via the real-time health monitoring platform 150 with the patient's permission. In other cases, the healthcare provider devices 120 communicate directly with the mobile health monitoring device(s) 112 to obtain the messages that include health monitoring information with the patient's permission.

The healthcare provider devices 120, when used by internal or external users, to access the real-time health monitoring platform 150 can view many records associated with many different patients (or users associated with client devices 110). Different levels of authorization can be associated with different internal and different external users to control which records the internal and external users have access. In some instances, only records associated with those patients to which a given internal or external user is referred, are made accessible and available to the given internal or external user device. Sometimes, a first internal or external user can refer a patient or records associated with the patient to a second internal or external user. In such circumstances, the second internal or external user becomes automatically authorized to access and view the patient's records that were referred by the first internal or external user.

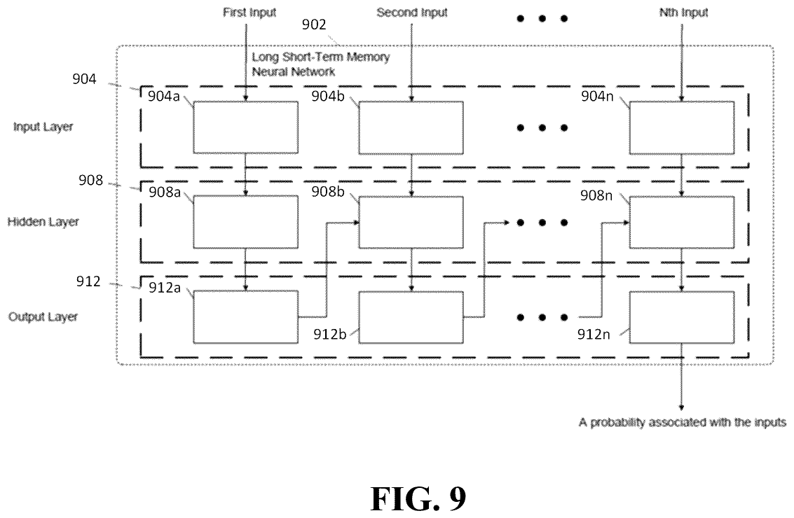

In some examples, the real-time health monitoring platform 150 (and specifically the outreach event selection system 156) can implement a machine learning technique or machine learning model, such as a neural network (discussed below in connection with FIG. 9). The machine learning technique can be trained to establish a relationship between a plurality of training mobile device class features and re-reading requests. In an example, the machine learning technique can be trained by obtaining a batch of training data that includes a first set of the plurality of training mobile device class features (e.g., types of blood pressure cuffs, types of weight scales, age of weight scales and/or blood pressure cuffs, etc.) associated with re-reading requests.

The first set of the plurality of training mobile device class features can be processed by the machine learning technique to generate an estimated need for a re-reading request. For example, the machine learning technique can generate a probability that a first reading is inaccurate based on one or more corresponding features of the first set of the plurality of training mobile device class features, and request a re-reading to obtain a second reading and verify the accuracy when the probability meets a re-reading threshold. A loss can be computed based on a deviation between the estimated need for a re-reading request (e.g., an estimated number of times that re-readings should be requested) and the re-reading request (e.g., the actual number of re-reading requests that were issued) associated with the first set of the plurality of training mobile device class features. Parameters of the machine learning technique can then be updated based on the computed loss. These training operations can be repeated for multiple batches of training data and/or until a stopping criterion is reached.

In this way, a given class of mobile health monitoring device 112 can be intelligently, dynamically and selectively instructed to re-read or re-collect a set of health monitoring information based on an output of the machine learning model. This can ensure that the readings or samples of the health monitoring information collected by the given class of mobile health monitoring device 112 is accurate when provided to the healthcare provider devices 120 or processed to determine a need for an outreach event or alert.

The network 130 may include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology. In some embodiments, the communication network can transmit the data in an encrypted format.

The healthcare provider devices 120 can be used to access pharmacy claims, medical data (e.g., medical information 230 stored in database 152), laboratory data and the like for one or more patients that the healthcare provider devices 120 are authorized to view. This patient information 210 can be maintained in a database 152 by the real-time health monitoring platform 150 or in a third-party database accessible to the real-time health monitoring platform 150 and/or the healthcare provider devices 120.

In some embodiments, the client devices 110 and the real-time health monitoring platform 150 can be communicatively coupled via an audio call (e.g., VOIP, Public Switched Telephone Network, cellular communication network, etc.) or via electronic messages (e.g., online chat, instant messaging, text messaging, email, and the like). While FIG. 1 illustrates a single client device 110 and a single healthcare provider device 120, it is understood that a plurality of such devices can be included in the system 100 in other embodiments. As used herein, the term “client device” may refer to any machine that interfaces to a communications network (such as network 130) to obtain resources from one or more server systems or other client devices.

The real-time health monitoring platform 150 can be an automated agent, e.g., on behalf of an organization. The automated agent can include a computing device that executes instructions stored therein or a device that executes instructions and provides a human machine interface. The automated agent can be associated with a medical group that includes the member. The automated agent can be an interactive voice response (IVR), a virtual online assistant, or a chatbot provided on a website. During a communication session between the user and the agent, the real-time health monitoring platform 150 identifies the member using initial context data (e.g., the phone number the member is calling from, the website login information inputted, automatic number identification (ANI), etc.) and retrieves the data on the member (e.g., member account information, name, address, insurance information, information on spouse and dependents, etc.) to be presented on the client device 110.

In some embodiments, the real-time health monitoring platform 150 includes an outreach event selection system 156. In some examples, the outreach event selection system 156 can receive data from one or more mobile health monitoring devices 112. Based on the received data, the outreach event selection system 156 selects a particular type of outreach event to perform. For example, the outreach event selection system 156 can receive health monitoring information of a particular type (or group, such as blood pressure, glucose, eye pressure, etc.) that fails to satisfy a criterion associated with the particular type of health monitoring information. For example, the outreach event selection system 156 can receive blood pressure measurement from a given mobile health monitoring device 112. The outreach event selection system 156 can compare the blood pressure measurement to a range of normal blood pressure measurements associated with an age group of the patient from which the blood pressure measurement was received. In response to determining that the blood pressure measurement is outside the normal range, the outreach event selection system 156 determines that the criterion associated with the blood pressure measurement fails to be satisfied. In such cases, the outreach event selection system 156 selects an outreach event that includes communicating with a healthcare provider device 120 to instruct a healthcare professional to contact the patient associated with the given mobile health monitoring device 112 from which the health monitoring information was received.

As another example, the given mobile health monitoring device 112 can transmit to the outreach event selection system 156 a timestamp indicating a last time that a particular type of health monitoring information was collected from the patient. The outreach event selection system 156 can compare the timestamp to a current time to measure a duration of time. If the duration of time exceeds a threshold (e.g., is more than 3 days indicating that the particular type of health monitoring information is stale), the outreach event selection system 156 can select an outreach event that triggers a notification or message to be presented on the client device 110 of the patient that is associated with or coupled to the mobile health monitoring device 112. The message can be transmitted by the outreach event selection system 156 via an SMS, automated phone call, chat, push notification or any other communication means. The message can inform the patient to collect the particular type of health monitoring information because the previous sample of the particular type of health monitoring information is stale. As another example, the outreach event can include transmission of a message to the healthcare provider device 120 instructing a healthcare professional to directly contact (e.g., by way of a phone call) the patient to instruct the patient to collect the particular type of health monitoring information.

The outreach event selection system 156 can store one or more rules for selecting the type of outreach event for different types of health monitoring information and types of patients and/or classes mobile health monitoring devices 112. The one or more rules can be automatically updated and/or generated with a machine learning model (e.g., a generative artificial intelligence model).

For example, the type of outreach event can be adjusted based on specific characteristics of the patient that is to receive the outreach event. That is, patient information can be analyzed to detect preferences, demographic information (e.g., age group) of the patient and so forth. The machine learning model can adjust the phrasing and/or type of outreach event based on the preferences and demographic information (e.g., patient specific information). Doing so can increase the effectiveness of the outreach, and increase the probability that the patient responds, follows and/or complies with instructions of the outreach event to provide health monitoring information. For example, suppose that the patient is a younger person (e.g., 25 years old) and the real-time health monitoring platform 150 is attempting to obtain blood pressure information. The phrasing of the outreach event can be adjusted to provide reasons why a younger person should be vigilant with blood pressure monitoring (e.g., “are you aware that young people who have moderate to high blood pressure that gradually rises over time may be at risk for poor brain health later in life? Let's monitor your blood pressure and develop a plan to reduce your blood pressure over time! Please take your blood pressure reading!”).

The outreach events can also be adjusted based on specific historical information of the patient that is to receive the outreach event. For example, the machine learning model can efficiently and quickly analyze historical information of the patient. The machine learning model can identify habits based on the historical information that the patient is more likely to embrace. For example, suppose that from the patient information (e.g., historical data), the machine learning model determines that the patient poorly monitors blood sugar levels when the “finger stick check” method is suggested (e.g., the patient routinely fails to provide sugar readings when the “finger stick check” method is suggested as part of the outreach). In the “finger stick check,” the patient pricks a fingertip with a small needle called a lancet to produce a blood drop. The drop of blood is placed against a test strip in a glucose meter, and the glucose meter displays blood glucose levels within the blood. Rather than suggesting the “finger stick check” method, examples can identify an alternative suggestion, and rather suggest that the patient obtain a continuous glucose monitoring (CGM) sensor and/or system to automatically provide glucose readings to the real-time health monitoring platform 150.

In some examples, the outreach event selection system 156 selects a given type of health monitoring information to collect from a patient. For example, a patient can input various medical information via a graphical user interface in the client device 110. The client device 110 can communicate with the real-time health monitoring platform 150 to provide the medical information about the patient. The real-time health monitoring platform 150 can then select a particular type of mobile health monitoring device 112 to monitor a health condition of the patient. For example, the medical information can indicate that the patient has high blood pressure. In such cases, the real-time health monitoring platform 150 can select a blood pressure monitoring device as the mobile health monitoring device 112 to use to periodically collect blood pressure measurements from the patient. As another example, the medical information can indicate that the patient suffers from diabetes. In such cases, the real-time health monitoring platform 150 can select a CGM device as the mobile health monitoring device 112 (e.g., a computing device) to use to periodically collect blood glucose measurements from the patient.

In some examples, a generative machine learning model can also be incorporated. The generative machine learning model can answer questions of the patient. For example, suppose that the patient receives an outreach event stating that a CGM device should be utilized to obtain glucose measurements, but has questions regarding how to use the CGM device, where to obtain the CGM device and so forth. The generative machine learning model can provide answers to the patient to increase the probability of patient compliance with instructions of the outreach event.

The outreach event selection system 156 instructs the mobile health monitoring device 112 (e.g., mobile IoT device) associated with the patient to collect the given type of health monitoring information. The outreach event selection system 156 can determine that the mobile health monitoring device 112 has failed to collect the given type of health monitoring information. For example, the outreach event selection system 156 can determine that an elapsed time since the last sample was received from the mobile health monitoring device 112 meets, exceeds or transgresses a threshold (e.g., the last sample was received more than 3 days ago, where 2 days is the threshold). In response, the outreach event selection system 156 generates an outreach event to request the given type of health monitoring information from the patient.

In some examples, the outreach event selection system 156 accesses previously collected health monitoring information associated with the patient. For example, the outreach event selection system 156 can obtain a history of health monitoring information collected from the patient over a past week, month, year or any other period. The history can include a variety of different types of health monitoring information, such as blood pressure measurements, heart rate measurements, blood glucose level measurement, and so forth. The outreach event selection system 156 searches the previously collected health monitoring information for the given type of health monitoring information. Specifically, the outreach event selection system 156 can be configured to monitor blood pressure measurements from the patient. In such cases, the outreach event selection system 156 can search or retrieve only blood pressure measurements or instances from the history of blood pressure measurements. The outreach event selection system 156 accesses a timestamp associated with the retrieved instance of the health monitoring information (e.g., the blood pressure measurement) and measures a duration of time between a current time and the timestamp. The outreach event selection system 156 compares the duration of time to a threshold and causes the outreach event to be generated in response to determining that the duration of time transgresses the threshold. In some cases, the outreach event selection system 156 selects a particular type of outreach event associated with a historical measurement (last measurement) of health information being received more than a threshold time than a current time.

In some examples, if health information is missing, the real-time health monitoring platform 150 can retrieve the health information from other sources rather than reaching out to a patient and/or selecting an outreach event. For example, longitudinal patient records can incorporate the internal/external health history of individuals, external events (e.g., manual patient reports, lab work, electronic medical records, etc.).

In some examples, the outreach event selection system 156 receives the given type of health monitoring information from the mobile device and determines that a value of the received given type of health monitoring information fails to satisfy a criterion. For example, the outreach event selection system 156 can determine that a blood pressure measurement falls outside of a normal range associated with the patient. In response, the outreach event selection system 156 instructs the mobile health monitoring device 112 to recollect or re-read the given type of health monitoring information.

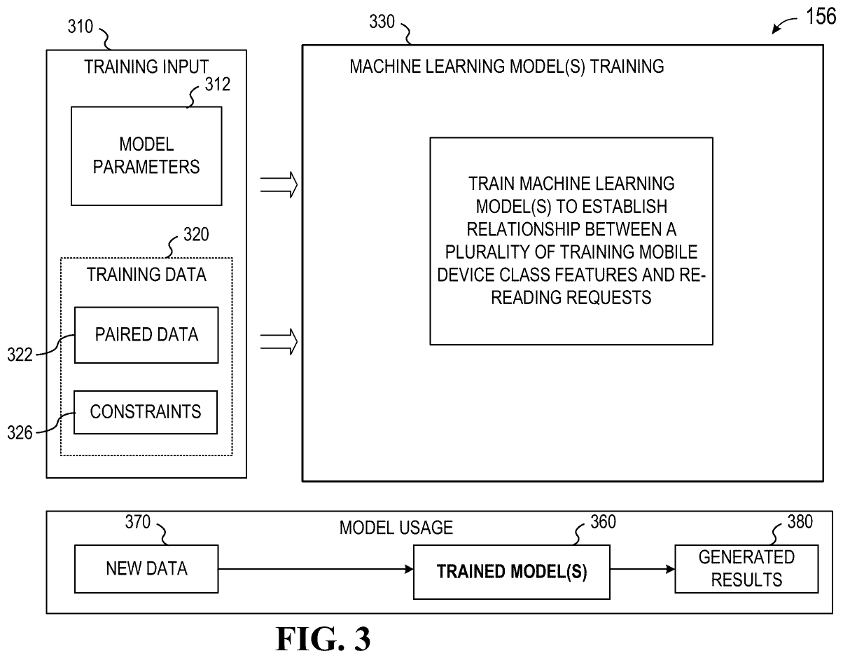

In some examples, as explained in more detail in connection with FIG. 3, the outreach event selection system 156 is trained based on training patient information features (e.g., patient health information, patient demographic information, patient in-network insurance coverage, patient out-of-network insurance coverage, patient location, or one or more treatment preferences) and their corresponding ground-truth types of service of care. The outreach event selection system 156 is trained to predict type of service of care for a given set of patient information features. The prediction can be used to select a type of service of care to recommend to a patient.

In some examples, the outreach event that is selected includes a reminder message transmitted to the client device 110 of the patient. Each reminder message includes a notification for the patient to provide or recollect the given type of health monitoring information using the mobile health monitoring device 112. The outreach event selection system 156 can continue to send reminder messages to the client device 110 over a period of time. The outreach event selection system 156 can determine that a quantity of the reminder messages previously sent to the client device 110 transgresses a threshold quantity (e.g., more than 3 reminders have been sent). The outreach event selection system 156 further determines that updated health monitoring information has not been received from the mobile health monitoring device 112 since the time the first reminder message was sent to the client device 110. In such cases, the outreach event selection system 156 can select a different outreach event, such as an outreach event that includes transmitting a message to a healthcare provider device 120 associated with a medical provider.

In some examples, the outreach event selection system 156 can determine a class associated with the mobile health monitoring device 112 from which a selected type of health monitoring information is received. The outreach event selection system 156 can maintain a list of different classes or types of health monitoring devices 112 that are associated with inaccurate readings or sample collections. The outreach event selection system 156 determines that the class associated with the mobile health monitoring device 112 matches or corresponds to the class or type of health monitoring devices 112 that are on the list. In response, the outreach event selection system 156 can select an outreach event that includes instructing the health monitoring devices 112 to re-collect the given type of health monitoring information. In some examples, the outreach event selection system 156 applies a trained neural network or machine learning model to the class of the mobile health monitoring device 112 to estimate a need for re-reading the health monitoring information. In response to obtaining a prediction from the machine learning model that the class of the mobile health monitoring device 112 requires re-reading, the outreach event selection system 156 selects an outreach event to instruct the mobile health monitoring device 112 to re-collect the sample of the health monitoring information.

In some examples, the outreach event selection system 156 determining a type associated with the patient associated with the mobile health monitoring device 112 from which the health monitoring information was received. The type can represent a demographic, medical information (condition), gender and/or age group of the patient. The outreach event selection system 156 can instruct the mobile device to re-collect the given type of health monitoring information based on determining the type associated with the patient. Specifically, certain types of patients may need to be requested to perform a re-reading (e.g., to recollect a sample of health monitoring information). As an example, a female patient that is 20 years old may be able hold her arm steady to collect a good sample of health monitoring information, such as a blood pressure measurement. For such a patient, a re-reading request may not be needed. As another example, a male patient that is 50 years old may not capable of holding his arm steady and so the sample of health monitoring information could be inaccurate. For such a patient, the outreach event selection system 156 can request that the mobile health monitoring device 112 re-collect the health monitoring information. As another example, a patient that has a certain medical condition, such as Parkinson's, may be incapable of holding their arm steady and so the sample of health monitoring information could be inaccurate. For such a patient, the outreach event selection system 156 can request that the mobile health monitoring device 112 re-collect the health monitoring information.

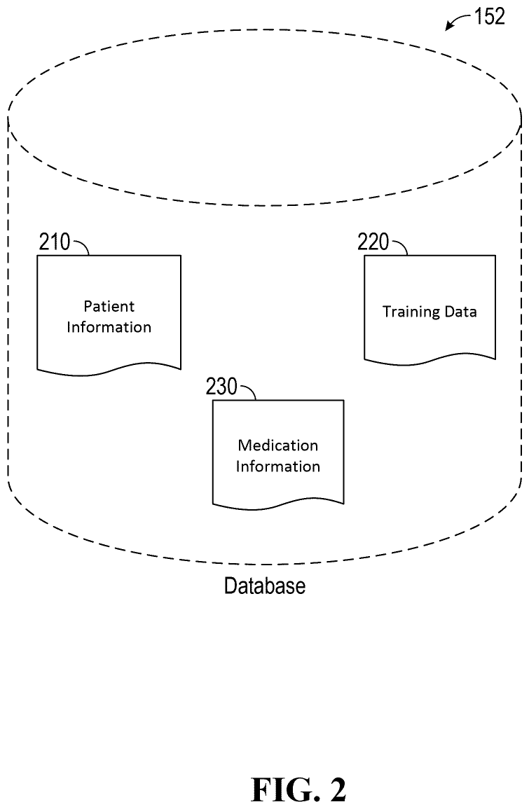

FIG. 2 is an example database 152 that may be deployed within the system of FIG. 1, according to some embodiments. The database 152 includes patient information 210, medication information 230, and training data 220. The patient information 210 can be generated by the real-time health monitoring platform 150. For example, the real-time health monitoring platform 150 can access one or more patient records from one or more sources, including pharmacy claims, benefit information, prescribing physician information, dispensing information (e.g., where and how the patient obtains their current medications), demographic information, prescription information including dose quantity and interval, and input from a patient received via a user interface presented on the client device 110 and so forth. The real-time health monitoring platform 150 can collect this information from the patient records and generates a patient features vector that includes this information.

The medication information 230 stores various medication related information (e.g., prescriptions, size of the medication or pills, compatible forms of dispensing information, temperature control information, mixing exclusion information, and so forth) for various medications. The medication information 230 can include a history of different samples or instances of health monitoring information collected from a variety of patients. The history can include timestamps for each health monitoring information indicating when that health monitoring information was collected from the mobile health monitoring device 112. The history can be stored in an encrypted manner. In this way, a patient can access their own health monitoring information and not health monitoring information of any other patient using their respectively assigned encryption/decryption key. The history of medical information can be accessed by the outreach event selection system 156 to determine how much time has elapsed since a last health monitoring information instance was collected and to select a particular type of outreach event to perform or trigger.

The training data 220 includes training sets of the plurality of training mobile device class features associated with re-reading requests. The training data 220 is used to train a machine learning model implemented by the outreach event selection system 156 to generate estimates of needs to perform re-reading requests. For example, the training data 220 can be built over time by identifying a first set of the plurality of training mobile device class features that are associated with a need to perform a re-reading.

FIG. 3 is a block diagram of an example outreach event selection system 156 that may be deployed within the system of FIG. 1, according to some embodiments. Training input 310 includes model parameters 312 and training data 320 (e.g., training data 220 (FIG. 2)) which may include paired training data sets 322 (e.g., input-output training pairs) and constraints 326. Model parameters 312 stores or provides the parameters or coefficients of corresponding ones of machine learning models. During training, these model parameters 312 are adapted based on the input-output training pairs of the training data sets 322. After the model parameters 312 are adapted (after training), the parameters are used by trained models 360 to implement the trained machine learning models on a new set of data 370.

Training data 320 includes constraints 326 which may define the constraints of a given patient information features. The paired training data sets 322 may include sets of input-output pairs, such as a pairs of a plurality of training mobile device class features and re-reading requests associated with the training mobile device class features. Some components of training input 310 may be stored separately at a different off-site facility or facilities than other components.

Machine learning model(s) training 330 trains one or more machine learning techniques based on the sets of input-output pairs of paired training data sets 322. For example, the machine learning model(s) training 330 may train the model parameters 312 (e.g., machine learning (ML) parameters) by minimizing a loss function based on one or more ground-truth type of needs to perform re-reading requests. The ML model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, the ML model can be applied to a training plurality of mobile device class features to estimate or generate a prediction of a need to perform a re-reading request. In some implementations, a derivative of a loss function is computed based on a comparison of the estimated need to perform a re-reading request and the ground truth re-reading requests and parameters of the ML model are updated based on the computed derivative of the loss function.

In one example, the ML model receives a batch of training data that includes a first set of the plurality of training mobile device class features together with a ground-truth re-reading requests need. The ML model generates a feature vector based on the first set of the plurality of training mobile device class features and generates a prediction of a need to perform a re-reading request. The prediction is compared with the ground truth need for re-reading and parameters of the ML model are updated based on the comparison.

The result of minimizing the loss function for multiple sets of training data trains, adapts, or optimizes the model parameters 312 of the corresponding ML models. In this way, the ML model is trained to establish a relationship between a plurality of training mobile device class features and re-reading requests.

After the machine learning model is trained, new data 370, including a class of a mobile device features are received. The trained machine learning technique may be applied to the new data 370 to generate results 380 including a prediction of a need to perform a re-reading request.

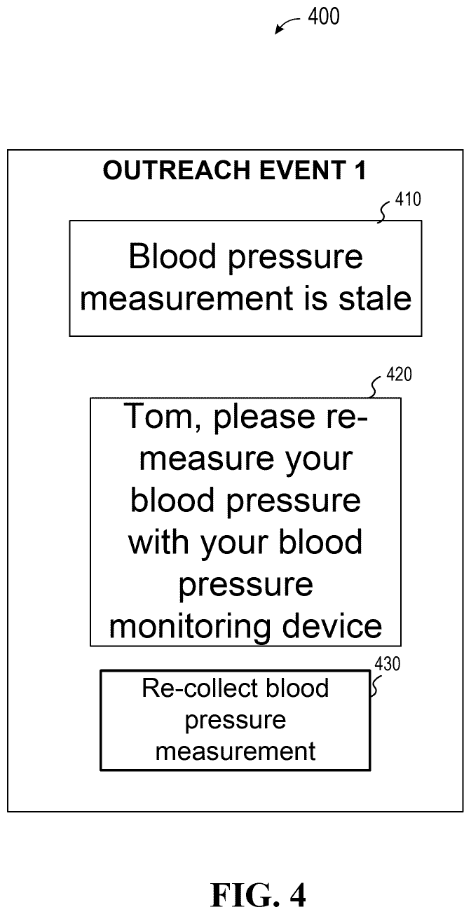

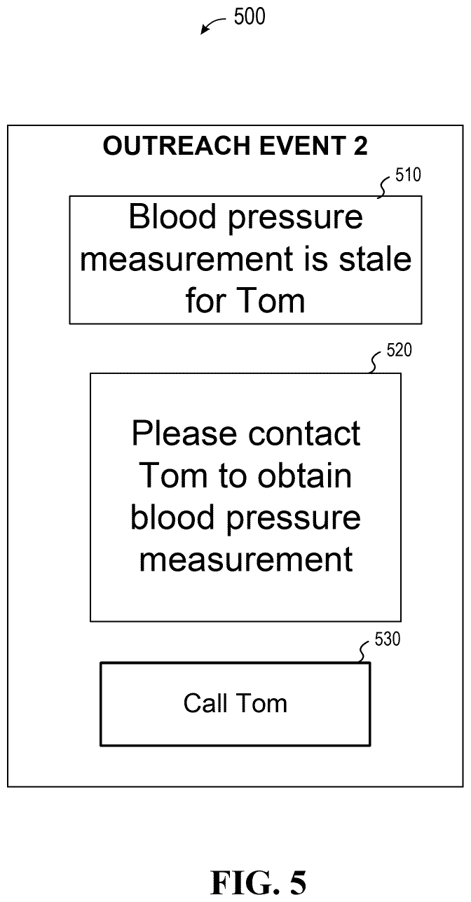

FIGS. 4 and 5 are example user interfaces 400 and 500 of the health monitoring system 100, according to example embodiments. For example, a client device 110 can provide medical information (patient information) associated with a patient to the outreach event selection system 156. To do so, the client device 110 can present a graphical user interface through which the patient inputs various patient information. The patient can also input a request for medical services, such as a request to schedule an appointment, fulfill a prescription, or view a list of medical providers.

In response to receiving, the request from the client device 110, the outreach event selection system 156 can select a type of health monitoring information to collect from the patient using the mobile health monitoring device 112 of the patient. The outreach event selection system 156 can determine that the mobile health monitoring device 112 has failed to collect a given type of health monitoring information and, in response, the outreach event selection system 156 can trigger a particular type of outreach event. In one example, the outreach event selection system 156 can trigger an outreach event that includes a reminder or notification provided to the client device 110 of the patient.

In some examples, in response to receiving the outreach event, the client device 110 can present a user interface 400 (FIG. 4). The user interface 400 includes an identification 410 of the outreach event (e.g., blood pressure measurement is stale—the elapsed time since the blood pressure measurement exceeds a threshold time). The user interface 400 includes a message 420 informing the patient to re-collect or to perform an action for collecting the health monitoring information, such as by wearing a blood pressure monitoring device. The user interface 400 includes an option 430 to re-sample or to collect the health monitoring information (e.g., an option to re-collect the blood pressure measurement). In response to receiving input that selects the option 430, the mobile health monitoring device 112 of the patient is activated and measures the health monitoring information. Once the mobile health monitoring device 112 completes measuring or collecting the health monitoring information, the mobile health monitoring device 112 transmits the information to the outreach event selection system 156 in real-time.

In some examples, the outreach event is transmitted to the healthcare provider device 120. In such cases, in response to receiving the outreach event, the healthcare provider device 120 can present a user interface 500 (FIG. 5). The user interface 500 includes an identification 510 of the outreach event (e.g., blood pressure measurement is stale—the elapsed time since the blood pressure measurement exceeds a threshold time) including an identification of the patient. The user interface 500 includes a message 520 informing the provider to contact the patient to instruct the patient to recollect the health monitoring information. The user interface 500 includes an option 530 to contact the patient. In response to receiving input that selects the option 530, the healthcare provider device 120 transmits a communication in real-time to the client device 110 of the patient, such as by way of an SMS, a phone call, a push notification, or other communication to instruct the patient to recollect the health monitoring information.

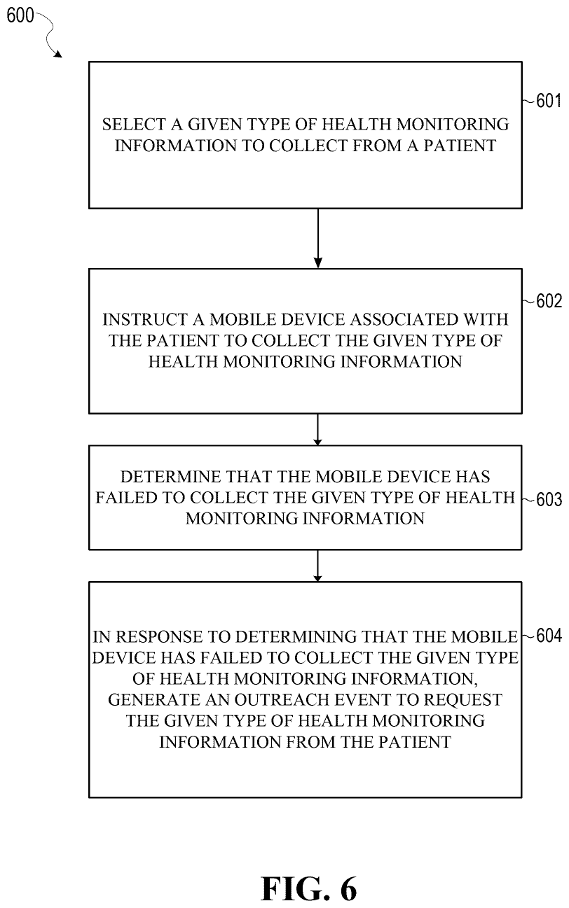

FIG. 6 is a flowchart illustrating example operations of the service of care type selection system in performing process 600, according to example embodiments. The process 600 may be embodied in computer-readable instructions for execution by one or more processors such that the operations of the process 600 may be performed in part or in whole by the functional components of the system 100; accordingly, the process 600 is described below by way of example with reference thereto. However, in other embodiments, at least some of the operations of the process 600 may be deployed on various other hardware configurations. Some or all of the operations of process 600 can be in parallel, out of order, or entirely omitted.

At operation 601, the system 100 selects a given type of health monitoring information to collect from a patient, as discussed above.

At operation 602, the system 100 instructs a mobile device associated with the patient to collect the given type of health monitoring information, as discussed above.

At operation 603, the system 100 determines that the mobile device has failed to collect the given type of health monitoring information, as discussed above.

At operation 604, the system 100, in response to determining that the mobile device has failed to collect the given type of health monitoring information, generates an outreach event to request the given type of health monitoring information from the patient, as discussed above.

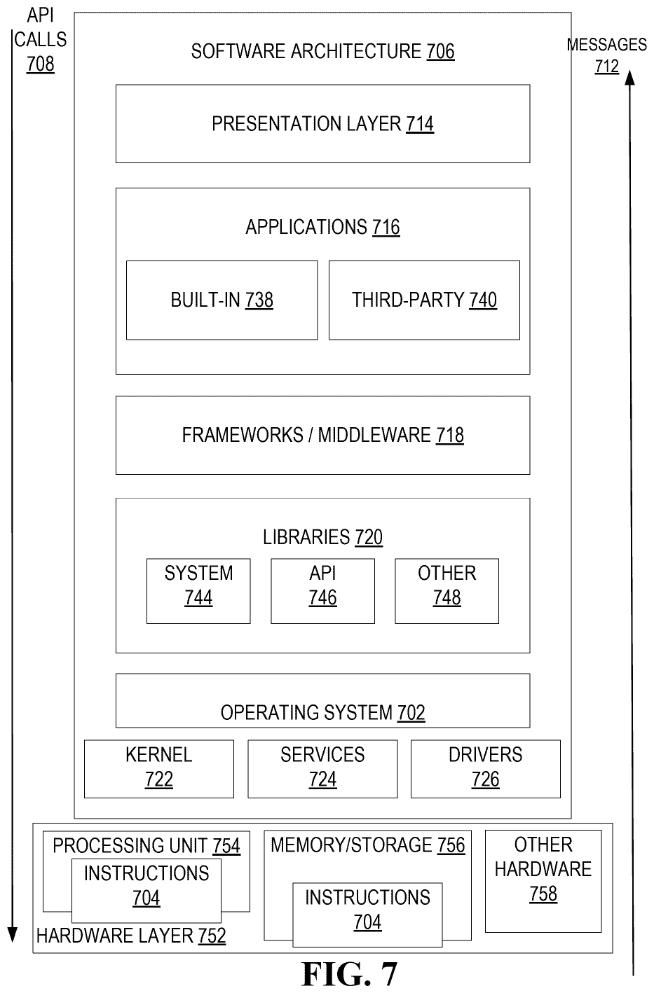

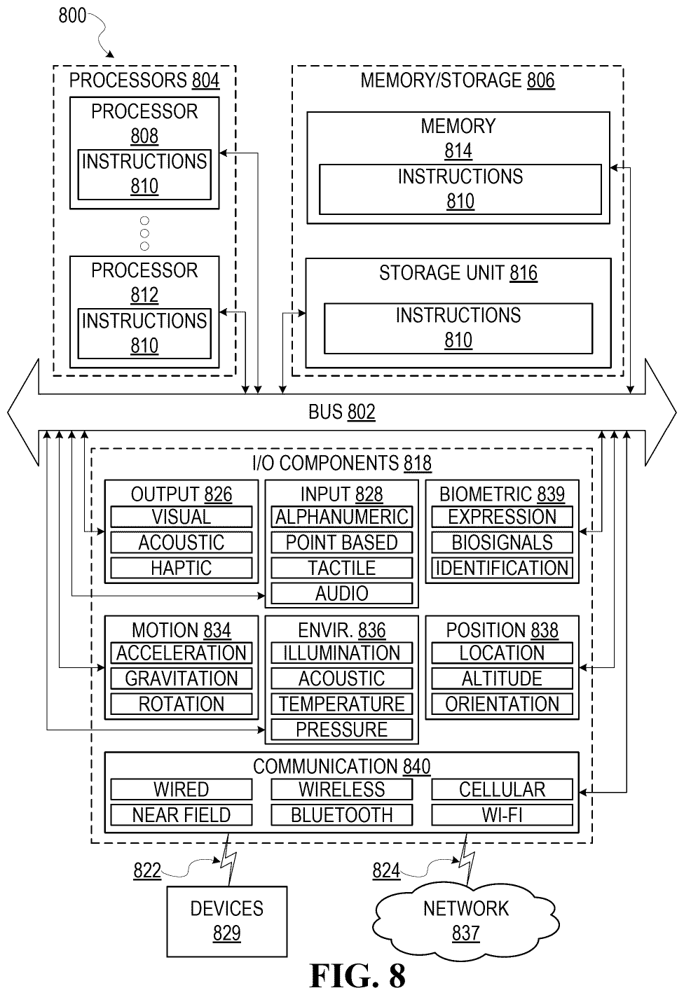

FIG. 7 is a block diagram illustrating an example software architecture 706, which may be used in conjunction with various hardware architectures herein described. FIG. 7 is a non-limiting example of a software architecture and it will be appreciated that many other architectures may be implemented to facilitate the functionality described herein. The software architecture 706 may execute on hardware such as machine 800 of FIG. 8 that includes, among other things, processors 804, memory 814, and input/output (I/O) components 818. A representative hardware layer 752 is illustrated and can represent, for example, the machine 800 of FIG. 8. The representative hardware layer 752 includes a processing unit 754 having associated executable instructions 704. Executable instructions 704 represent the executable instructions of the software architecture 706, including implementation of the methods, components, and so forth described herein. The hardware layer 752 also includes memory and/or storage devices memory/storage 756, which also have executable instructions 704. The hardware layer 752 may also comprise other hardware 758. The software architecture 706 may be deployed in any one or more of the components shown in FIG. 1. The software architecture 706 can be utilized to apply a machine learning technique or model to generate a prediction of a need to perform re-reading for a particular class of mobile health monitoring device 112 of a patient. The software architecture 706 can be utilized to select an outreach event to perform from a plurality of different types of outreach events based on failure to collect a given type of health monitoring information from a mobile health monitoring device 112.

In the example architecture of FIG. 7, the software architecture 706 may be conceptualized as a stack of layers where each layer provides particular functionality. For example, the software architecture 706 may include layers such as an operating system 702, libraries 720, frameworks/middleware 718, applications 716, and a presentation layer 714. Operationally, the applications 716 and/or other components within the layers may invoke API calls 708 through the software stack and receive messages 712 in response to the API calls 708. The layers illustrated are representative in nature and not all software architectures have all layers. For example, some mobile or special purpose operating systems may not provide a frameworks/middleware 718, while others may provide such a layer. Other software architectures may include additional or different layers.

The operating system 702 may manage hardware resources and provide common services. The operating system 702 may include, for example, a kernel 722, services 724, and drivers 726. The kernel 722 may act as an abstraction layer between the hardware and the other software layers. For example, the kernel 722 may be responsible for memory management, processor management (e.g., scheduling), component management, networking, security settings, and so on. The services 724 may provide other common services for the other software layers. The drivers 726 are responsible for controlling or interfacing with the underlying hardware. For instance, the drivers 726 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 720 provide a common infrastructure that is used by the applications 716 and/or other components and/or layers. The libraries 720 provide functionality that allows other software components to perform tasks in an easier fashion than to interface directly with the underlying operating system 702 functionality (e.g., kernel 722, services 724 and/or drivers 726). The libraries 720 may include system libraries 744 (e.g., C standard library) that may provide functions such as memory allocation functions, string manipulation functions, mathematical functions, and the like. In addition, the libraries 720 may include API libraries 746 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 720 may also include a wide variety of other libraries 748 to provide many other APIs to the applications 716 and other software components/devices.

The frameworks/middleware 718 (also sometimes referred to as middleware) provide a higher-level common infrastructure that may be used by the applications 716 and/or other software components/devices. For example, the frameworks/middleware 718 may provide various graphic user interface functions, high-level resource management, high-level location services, and so forth. The frameworks/middleware 718 may provide a broad spectrum of other APIs that may be utilized by the applications 716 and/or other software components/devices, some of which may be specific to a particular operating system 702 or platform.

The applications 716 include built-in applications 738 and/or third-party applications 740. Examples of representative built-in applications 738 may include, but are not limited to, a contacts application, a browser application, a book reader application, a location application, a media application, a messaging application, and/or a game application. Third-party applications 740 may include an application developed using the ANDROID™ or IOS™ software development kit (SDK) by an entity other than the vendor of the particular platform, and may be mobile software running on a mobile operating system such as IOS™ ANDROID™, WINDOWS® Phone, or other mobile operating systems. The third-party applications 740 may invoke the API calls 708 provided by the mobile operating system (such as operating system 702) to facilitate functionality described herein.

The applications 716 may use built-in operating system functions (e.g., kernel 722, services 724, and/or drivers 726), libraries 720, and frameworks/middleware 718 to create UIs to interact with users of the system. Alternatively, or additionally, in some systems, interactions with a user may occur through a presentation layer, such as presentation layer 714. In these systems, the application/component “logic” can be separated from the aspects of the application/component that interact with a user.

FIG. 8 is a block diagram illustrating components of a machine 800, according to some example embodiments, able to read instructions from a machine-readable medium (e.g., a machine-readable storage medium) and perform any one or more of the methodologies discussed herein. Specifically, FIG. 8 shows a diagrammatic representation of the machine 800 in the example form of a computer system, within which instructions 810 (e.g., software, a program, an application, an applet, an app, or other executable code) for causing the machine 800 to perform any one or more of the methodologies discussed herein may be executed. For example, the instructions 810 may be executed by the system 100 to process a combination of patient information features with a trained machine learning model to predict a need to perform a re-reading request for a class of mobile health monitoring device 112.

As such, the instructions 810 may be used to implement devices or components described herein. The instructions 810 transform the machine 800 (e.g., a general, non-programmed machine) into a particular machine programmed to carry out the described and illustrated functions in the manner described. In alternative embodiments, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by machine 800. Further, while only a single machine 800 is illustrated, the term “machine” shall also be taken to include a collection of machines that individually or jointly execute the instructions 810 to perform any one or more of the methodologies discussed herein.

The machine 800 may include processors 804, memory/storage 806, and I/O components 818, which may be configured to communicate with each other such as via a bus 802. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 808 and a processor 812 that may execute the instructions 810. The term “processor” is intended to include multi-core processors 804 that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiple cores, or any combination thereof.

The memory/storage 806 may include a memory 814, such as a main memory, or other memory storage, database 152, and a storage unit 816, both accessible to the processors 804 such as via the bus 802. The storage unit 816 and memory 814 store the instructions 810 embodying any one or more of the methodologies or functions described herein. The instructions 810 may also reside, completely or partially, within the memory 814, within the storage unit 816, within at least one of the processors 804 (e.g., within the processor's cache memory), or any suitable combination thereof, during execution thereof by the machine 800. Accordingly, the memory 814, the storage unit 816, and the memory of processors 804 are examples of machine-readable media.

The I/O components 818 may include a wide variety of components to receive input, provide output, produce output, transmit information, exchange information, capture measurements, and so on. The specific I/O components 818 that are included in the machine 800 will depend on the type of machine. For example, portable machines such as mobile phones will likely include a touch input device or other such input mechanisms, while a headless server machine will likely not include such a touch input device. It will be appreciated that the I/O components 818 may include many other components that are not shown in FIG. 8. The I/O components 818 are grouped according to functionality merely for simplifying the following discussion and the grouping is in no way limiting. In various example embodiments, the I/O components 818 may include output components 826 and input components 828. The output components 826 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 828 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 818 may include biometric components 839, motion components 834, environmental components 836, or position components 838 among a wide array of other components. For example, the biometric components 839 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 818 may include communication components 840 operable to couple the machine 800 to a network 837 or devices 829 via coupling 824 and coupling 822, respectively. For example, the communication components 840 may include a network interface component or other suitable device to interface with the network 837. In further examples, communication components 840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 829 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geo-location, location via Wi-Fi® signal triangulation, location via detecting a NFC beacon signal that may indicate a particular location, and so forth.

FIG. 9 is a functional block diagram of an example neural network 902 that can be used for the inference engine or other functions (e.g., engines) as described herein to produce a predictive model. The predictive model can identify a need to perform a re-reading based on a class of mobile health monitoring device 112. In an example, the neural network 902 can be a LSTM neural network. In an example, the neural network 902 can be a recurrent neural networks (RNN). The example neural network 902 may be used to implement the machine learning as described herein, and various implementations may use other types of machine learning networks. The neural network 902 includes an input layer 904, a hidden layer 908, and an output layer 912. The input layer 904 includes inputs 904a, 904b . . . 904n. The hidden layer 908 includes neurons 908a, 908b . . . 908n. The output layer 912 includes outputs 912a, 912b . . . 912n.

Each neuron of the hidden layer 908 receives an input from the input layer 904 and outputs a value to the corresponding output in the output layer 912. For example, the neuron 908a receives an input from the input 904a and outputs a value to the output 912a. Each neuron, other than the neuron 908a, also receives an output of a previous neuron as an input. For example, the neuron 908b receives inputs from the input 904b and the output 912a. In this way the output of each neuron is fed forward to the next neuron in the hidden layer 908. The last output 912n in the output layer 912 outputs a probability associated with the inputs 904a-904n. Although the input layer 904, the hidden layer 908, and the output layer 912 are depicted as each including three elements, each layer may contain any number of elements. Neurons can include one or more adjustable parameters, weights, rules, criteria, or the like.

In various implementations, each layer of the neural network 902 must include the same number of elements as each of the other layers of the neural network 902. For example, training mobile device class features may be processed to create the inputs 904a-904n. The neural network 902 may implement a model to produce re-reading request prediction or selection for at least one of the mobile device class features. More specifically, the inputs 904a-904n can include mobile device class features (binary, vectors, factors or the like) stored in storage devices.

The mobile device class features can be provided to neurons 908a-908n for analysis and connections between the known facts. The neurons 908a-908n, upon finding connections, provides the potential connections as outputs to the output layer 912, which determines a need to perform a re-reading or to select an outreach event of a particular type from many types of outreach events.

The neural network 902 can perform any of the above calculations. The output of the neural network 902 can be used to trigger an outreach event including providing a notification. For example, the notification can be provided to a PBM, health plan manager, pharmacy, physician, caregiver, and/or a patient.

In some embodiments, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 904a is connected to each of neurons 908a, 908b . . . 908n.

### Analysis of Health Data Generation of Action Plans Examples

The aforementioned embodiments described various health monitoring processes to monitor health. For example, health monitoring information can be accurately and reliably obtained through various mechanisms (e.g., an automatically generated outreach event). The below embodiments further describe the analysis of the health monitoring information and the generation of action plans to mitigate health conditions identified based on the health monitoring information.

Patient health information can be difficult to track, monitor, and analyze. For example, a patient can have inaccurate blood pressure readings at a healthcare provider's office due to stresses of the environment (e.g., “white coat syndrome”). Furthermore, patients are typically at a healthcare provider's offices for a short period of time which limits the ability of healthcare providers to track certain health data over a longer period of time. Generating a health plan based on time-limited data can cause inaccuracies (e.g., misdiagnosis). Furthermore, retesting a patient at the health provider's office can cause significant delays due to scheduling difficulties, and can cause delays in appropriate treatments being administered. In some cases, the healthcare provider can receive and analyze remotely collected health data. Despite the remote collection of health data, significant challenges remain. For example, remotely collected data can lack data due to patient error (e.g., noncompliance). Moreover, the above process consumes significant time and resources.

Even when the health data is received, a healthcare provider can misdiagnose or administer a suboptimal treatment plan. For example, a healthcare provider has limited training and limited experience. Furthermore, the healthcare provider has a limited capacity to utilize different types of sensor data. Moreover, healthcare providers employ a subjective decision-making process resulting in misdiagnosis as well as suboptimal and/or delayed treatment plans. Thus, significant challenges exist with respect to health condition diagnosis and treatment.

The disclosed embodiments provide systems and methods to monitor patient health in real-time using mobile monitoring devices or equipment, such as Internet of Things (IoT) devices. The disclosed embodiments automatically receive health data (e.g., patient self-reported data such as surveys in different forms, patient associated inputs from health care professionals, etc.) about a patient from a patient device and/or patient databases, analyze the health data, generate an action plan and provide the action plan to the patient (or enact the action plan) substantially in real-time. Doing so reduces latency, reduces utilization of resources, and improves patient outcomes. For example, some embodiments implement a machine learning prediction model that is trained based on a large dataset to effectively identify health conditions, probabilities of health conditions occurring in the future, and action plans to mitigate current health conditions and/or the potential future health conditions. Thus, embodiments can leverage the enhanced abilities of the machine learning prediction model to effectively reduce resource consumption while enhancing patient outcomes.

In this way, the disclosed embodiments improve the overall process of providing medical care and specifically improve the efficiency at which health information is received and analyzed to generate action plans. As a result, latency and resource consumption is reduced. For example, embodiments herein can reduce the number of times a patient visits a healthcare provider.

Furthermore, embodiments herein can implement artificial intelligence to train on sizeable and comprehensive datasets to diagnose and generate action plans. Doing so enables several technical enhancements, including reducing or removing human subjectivity from decision making (i.e., diagnosis and treatment) enabling a consistent approach with superior outcomes. Moreover, embodiments herein can receive data from various heterogeneous sensors (e.g., temperature sensor, motion sensor, etc.) that would not be utilized by a typical healthcare provider. Furthermore, such embodiments can predictively model future outcomes based on health data to predict whether a health condition will occur in the future, and actively attempt to prevent the health condition from occurring.

Thus, embodiments herein result in several technical enhancements. By reducing or removing human subjectivity, health conditions are more accurately assessed. Based on the more accurate assessment, the health conditions can be addressed to quickly minimize patient impact, hospital resources (e.g., addressing a health condition earlier results in better outcomes with reduced consumption of hospital resources), while also enhancing patient experience. For example, once a health condition is identified in a patient, an action plan is automatically selected to mitigate the health condition and/or reduce the probability of the health condition occurring.

Moreover, yet another technical enhancement is faster speed and higher efficiency of the diagnosis and treatment, while also conserving computing resources by automatically, dynamically, and intelligently selecting efficient and effective health plans which are dynamically adjustable to enable better outcomes. As such, some examples enhance the technical areas of computerized health condition prediction and/or health condition identification, as well as action plan generation based on the health condition prediction and/or health condition identification. Yet another technical enhancement can include using a rigorous, computerized process to perform tasks (e.g., an efficient way to diagnose health conditions and generate health plans based on historical data associated with a plurality of patients and sensor data from a plurality of different sensors that measure various features such as environmental conditions) that were not previously able to be performed by humans in a practical or accurate manner. Some examples can further include referrals to a physician when a condition becomes either incalculable due to being out of a permissible range or other unknown factors, and/or the health condition expanding beyond a digital experience and into immediate health care.

In some examples, embodiments herein further encompass behavioral cases. For example, patient behaviors can be tracked and monitored as part of the health information/health data. Action plans to modify behaviors of a patient can be generated.

Furthermore, while some embodiments describe specific use cases, it should be understood that these are merely examples. It should be understood that embodiment are more expansive beyond behavioral. Embodiments can be applied to diagnosing health conditions and health treatments of any disease condition.

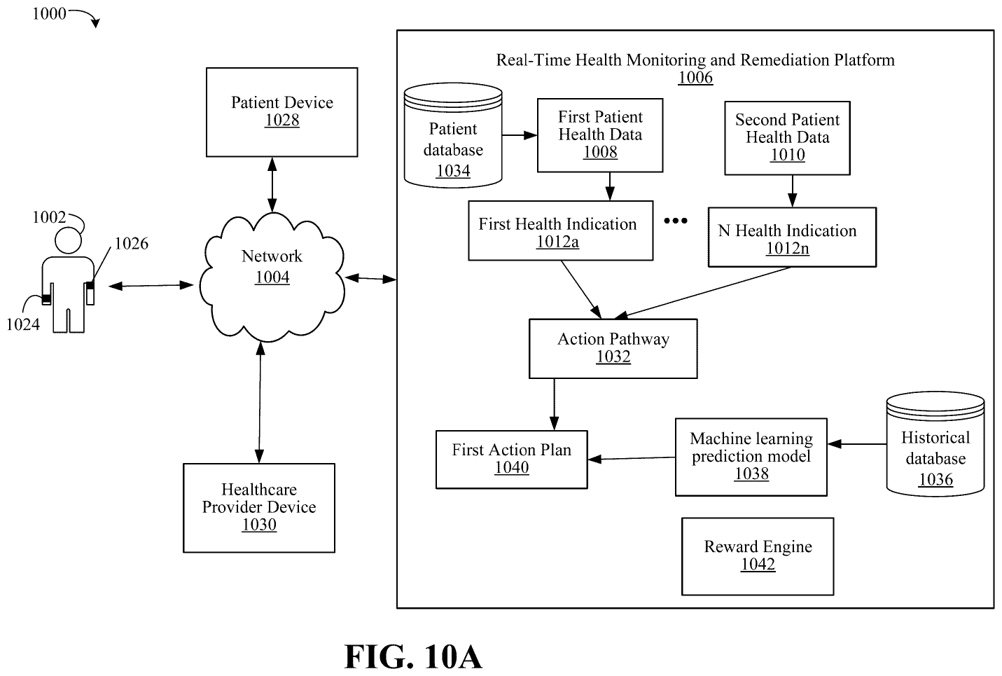

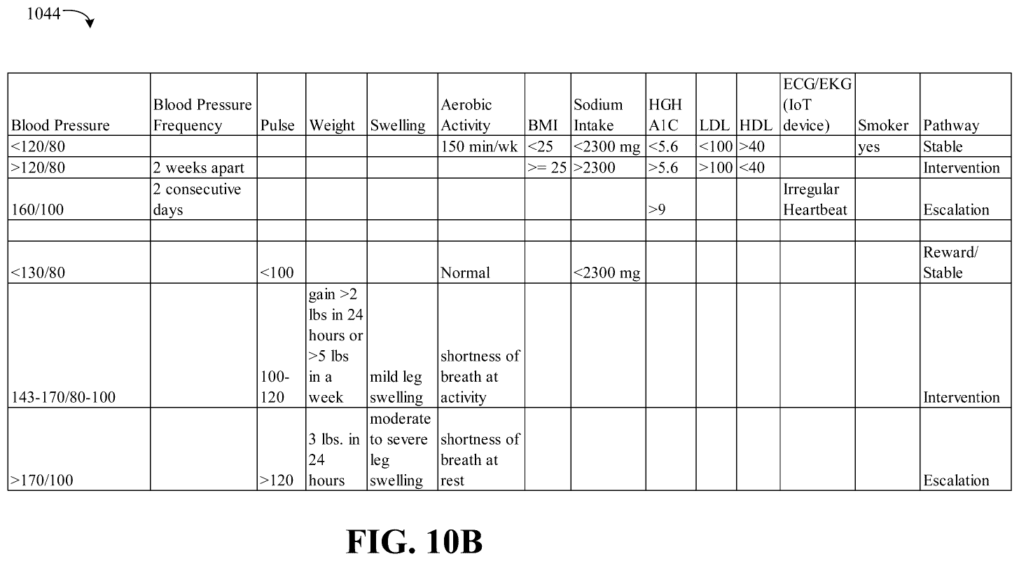

FIG. 10A illustrates a health monitoring, identification, and treatment process 1000. FIG. 10A can be readily incorporated into and/or operate with any of the embodiments described herein, for example the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), and neural network 902 (FIG. 9).

A healthcare provider device 1030, a real-time health monitoring platform (RTHMRP) 1006, a blood pressure cuff 1026, smart watch 1024, and patient device 1028 are communicatively coupled over a network 1004 (e.g., Internet, telephony network, etc.). Some embodiments further monitor patient health based on patient self-reported data such as surveys in different forms, patient-associated inputs from health care professionals, etc. to modify behaviors of the patient 1002.

As illustrated, several health monitoring devices are attached to a patient 1002 (e.g., a user). For example, each of the blood pressure cuff 1026 and smart watch 1024 includes hardware and/or software configured to collect and/or measure a particular type of health monitoring information. It will be understood that while a blood pressure cuff 1026 and smart watch 1024 are specifically discussed in the below example, various other types of health monitoring devices can operate with the embodiments described herein. Various other types of health monitoring devices include, e.g., a smart scale, an exercise bracelet, glucometer, and/or an ECG device. In some examples, and as noted above, if a given group of monitoring information (e.g., blood pressure, heart rate, etc.) is determined as being unavailable to be provided by one or more of the blood pressure cuff 1026 or the smart watch 1024 (e.g., a first computing device), examples generate an outreach event to request the given group of monitoring information from the patient 1002.

The blood pressure cuff 1026 monitors a blood pressure and heart rate of the patient 1002, while the smart watch 1024 monitors a heart rate (e.g., resting heart rate, heart rate during walking, etc.) of the patient 1002, maximal oxygen consumption (i.e., VO2 max) of the patient 1002, fitness activity (e.g., number of steps, minutes of cardiovascular activity, etc.) of the patient 1002, sleep activity of the patient 1002, electrocardiogram activity of the patient 1002, menstrual cycles of the patient 1002, etc. The blood pressure cuff 1026 and smart watch 1024 can be connected to the network 1004 directly, or through patient device 1028 (e.g., a computing device such as a laptop, mobile device, palmtop computer, etc.) of the patient 1002. Various other types of health data can also be generated by other health monitoring devices. For example, other types of health data can include aerobic activity information, body mass index (BMI) information, sodium intake information, arrhythmia information, and/or glucose information. In some examples, the patient 1002 can manually input health data (e.g., sodium intake information) into the patient device 1028 for transmission to the RTHMRP 1006 and storage as patient data. In some examples, recorded information can be provided to the RTHMRP 1006 and stored as patient data, and includes a physician diagnosis or other health care professional collected data.

The patient device 1028 can include a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a patient can use to access the network 1004 or the RTHMRP 1006. The patient device 1028 can directly receive health data from the blood pressure cuff 1026 and the smart watch 1024, and transmit the same to the RTHMRP 1006.

In some examples, the health data from the patient device 1028, the blood pressure cuff 1026, and/or the smart watch 1024 can be transmitted over a secure connection to the RTHMRP 1006. After establishing the secure connection, the patient device 1028, the blood pressure cuff 1026, and/or the smart watch 1024 can be configured to exchange encrypted communications in real time with each other, the healthcare provider device 1030, and/or the RTHMRP 1006. In this way, a patient's health monitoring information remains securely stored on the patient's device 1028 and cannot be compromised. This ensures that the health monitoring information is privately maintained. In some cases, functionality of one or more of the blood pressure cuff 1026 and smart watch 1024 can be implemented by the patient device 1028. This can avoid the need to use a separate hardware device to collect a given type of health monitoring information.

The healthcare provider device 1030 can include the same or similar functionality as the blood pressure cuff 1026, the smart watch 1024, and the patient device 1028 to generate health data of the patient 1002. In some embodiments, the healthcare provider device 1030 can receive communications from and/or access the RTHMRP 1006, the blood pressure cuff 1026, the smart watch 1024, and/or the patient device 1028.

In some embodiments, the healthcare provider device 1030 is used by “internal” patients. Internal patients are personnel, such as physicians, clinicians, healthcare providers, health-related coaches, pharmacy benefit manager (PBM) operators, pharmacists, specialty pharmacy operators or pharmacists, or the like that are associated with or employed by an organization that provides the RTHMRP 1006. In some cases, the healthcare provider device 1030 is used by “external” patients. External patients are personnel, such as physicians, clinicians, and health-related coaches that are associated with or employed by a different (external) organization than that which provides the RTHMRP 1006. In some cases, the healthcare provider device 1030 transmits real-time data, such as messages, to the RTHMRP 1006 to provide health measurements and analysis of the patient 1002. For example, such health measurements and analysis can be collected and entered into the healthcare provider's Electronic Health Record platform (EHR) and sent through a vendor to the patient longitudinal patient record, and then consumed by the RTHMRP 1006 to be stored as patient data.

The RTHMRP 1006 can analyze the health data to identify trends and occurrences of health matters. To do so, the RTHMRP 1006 determines associations between health-related data and first-N health indications 1012a-1012n. The first-N health indications 1012a-1012n can be different types of health classifications such as normal blood pressure, elevated blood pressure, Stage 1 high blood pressure, Stage 2 high blood pressure, pre-diabetic, no diabetes, diabetic, high cholesterol, normal cholesterol, physical activity above a threshold, physical activity below a threshold, smoker, non-smoker, etc. Based on the associations, the RTHMRP 1006 can identify health conditions and recommend action plans to remedy the health conditions.

For example, the RTHMRP 1006 can receive second patient health data 1010 from the blood pressure cuff 1026 (e.g., the second patient health data 1010 is from the given group of monitoring information). The RTHMRP 1006 can analyze the second patient health data 1010 and identify a health measurement contained in the second patient health data 1010. The health measurement can be a blood pressure (e.g., blood pressure information) reading including diastolic reading of 88 and systolic measurements of 138 (138/88). It is worthwhile to note that while blood pressure is described herein as the health measurement, embodiments can operate similarly with any other type of health related measurement (e.g., heart rate information, cholesterol information, breathing information, etc.).

The RTHMRP 1006 associates the second patient health data 1010 with N health indication 1012n based on the health measurement of the second patient health data 1010. For example, the health measurement of 138/88 can be considered slightly elevated blood pressure (stage I hypertension). The N health indication 1012n can be an indication of slightly elevated blood pressure, and thus the second patient health data 1010 is associated with the N health indication 1012n.

In some examples, blood pressure can fluctuate significantly over a period of time based on sleep patterns, diet, stress, exercise, mood, etc. Thus, one elevated blood pressure can be an anomaly rather than indicative of a chronic condition. Therefore, the RTHMRP 1006 can provide a request (e.g., a re-read request as described above) to the patient device 1028 via an application on the patient device 1028 and/or the blood pressure cuff 1026 to obtain further blood pressure readings over a period of time. The RTHMRP 1006 can provide instructions to obtain the blood pressure readings at certain times of day, in response to certain conditions being detected and/or over periodically over a period of time. The application can be an extension of the RTHMRP 1006.

For example, and as noted above, stress can affect blood pressure readings. Thus, the RTHMRP 1006 can instruct the blood pressure cuff 1026 and/or the patient 1002 via the application to obtain a blood pressure reading when a patient's heart rate as measured by the smart watch 1024 is below a certain threshold and/or within a range of the resting heart rate as measured by smart watch 1024. For example, the RTHMRP 1006 can identify the heart rate from the smart watch 1024, and provide an instruction to the blood pressure cuff 1026 and/or the patient 1002 via the application to obtain a blood pressure reading when the heart rate is below the certain threshold and/or within the range of the resting heart rate. If the patient 1002 is not wearing the blood pressure cuff 1026, the patient 1002 can be instructed through the patient device 1028 and/or the patient 1002 via the application to put on the blood pressure cuff 1026, and then obtain the reading. In some embodiments, the blood pressure cuff 1026 can interact directly with the smart watch 1024 to determine when to obtain a blood pressure reading, for example based on an instruction from the smart watch 1024 and/or a heart rate indication from the smart watch 1024. Furthermore, several blood pressure readings can be obtained and provided to the RTHMRP 1006 as second patient health data 1010.

Thus, the RTHMRP 1006 can thus receive multiple health measurements (e.g., blood pressure readings) from the blood pressure cuff 1026, and store the multiple health measurements as part of the second patient health data 1010. The RTHMRP 1006 can then determine whether a certain number of the health measurements are associated with the N health indication 1012n (stage I hypertension) by being above a threshold. If so, the second patient health data 1010 is associated with the N health indication 1012n since many elevated blood pressure readings reliably indicate the persistence of stage I hypertension. Otherwise, if the number of health measurements is below the threshold, the RTHMRP 1006 determines that any elevated blood pressure readings are abnormalities rather than being indicative of a chronic health condition. In such a case, the second patient health data 1010 would not be associated with the N health indication 1012n, and would be associated with another health indication from the first-N health indications 1012a-1012n indicating that the patient 1002 has normal blood pressure.

Thus, the RTHMRP 1006 can verify whether the second patient health data 1010 is an abnormality based on whether several health measurements in the second patient health data 1010 (i.e., blood pressure readings) are within elevated boundaries. In this example, the RTHMRP 1006 determines that the health measurement(s) (i.e., blood pressure reading(s)) contained in the second patient health data 1010 is elevated, and therefore that the second patient health data 1010 is associated with the N health indication 1012n.

The RTHMRP 1006 can further select and extract first patient health data 1008 from a patient database 1034. The patient database 1034 can store numerous healthcare records from different patients. The RTHMRP 1006 can look-up the first patient health data 1008 based on identification information (e.g., a name, social security number, etc.) of the patient 1002. The first patient health data 1008 can include, e.g., medical records of the patient 1002, family history of the patient 1002, a geographic location of the patient 1002, self-reported data of the patient 1002, surveys in different forms answered by the patient 1002, inputs related to the patient 1002 that are collected from healthcare professionals, and demographic information of the patient 1002. The first patient health data 1008 can for example, further include a family history of cardiovascular illness, a geographic location where cardiovascular issues are prevalent, externally derived disease diagnoses that are derived for example by external engines to generate an indicator and the indicator is stored into the longitudinal patient record, and previous medical measurements taken at various settings and with various devices (e.g., provider's office, previous medical readings, etc.). It is worthwhile to note that the patient database 1034 can be encrypted to protect patient anonymity, and decrypted on a patient-by-patient basis and in response to a requesting user being authenticated.

The first patient health data 1008 is associated with the first health indication 1012a. For example, the first patient health data 1008 can include a health measurement (e.g., a number of family members with cardiovascular issues, previous readings taken with other devices, etc.). The first patient health data 1008 can be associated with the first health indication 1012a based on the health measurement of the first patient health data 1008, which indicates a presence of the first health indication 1012a. For example, the first health indication 1012a can be an indication of pre-disposition towards hypertension due to family genetics. In such a case, if the number of family members indicated in the first patient health data 1008 is above a threshold, the first patient health data 1008 is associated with the first health indication 1012a. Otherwise, the first patient health data 1008 can be associated with a different health indication from the first-N health indications 1012a-1012n indicating no genetic predisposition towards hypertension. In this example, the first patient health data 1008 indicates a predisposition towards hypertension, and thus is associated with the first health indication 1012a. In some embodiments, the first and second patient health data 1008, 1010 can be associated to a same health indication (e.g., hypertension) of the first health indication 1012a-N health indication 1012n depending on the nature of the first health indication 1012a-N health indication 1012n.

The RTHMRP 1006 can then identify an action pathway 1032 for the patient 1002. The action pathway 1032 can indicate a level of care that is needed for the patient 1002. For example, the action pathway 1032 is one of a stable pathway, an intervention pathway, and an escalation pathway, although other pathways can be included based on the specific nature of the health condition (e.g., behavioral condition). A stable pathway is selected when the first patient health data 1008 and the second patient health data 1010 indicates that the patient 1002 is stable and therefore only routine health checkups are needed. A stable pathway can be characterized by a blood pressure of less than 120/80, moderate aerobic activity of 150 minutes or more per week, a BMI of less than 25, daily sodium intake of less than 2300, hgb A1C of less than 5.6, low-density lipoprotein (LDL) of less than 100, high-density lipoprotein (HDL) of greater than 40 and being a non-smoker (e.g., readings that are within normal ranges).

The intervention pathway is selected when the first patient health data 1008 and the second patient health data 1010 indicate that the patient 1002 has emerging health conditions and/or has a health condition, but does not need immediate medical attention (e.g., the health condition is not exigent). The intervention pathway can be characterized by a blood pressure within the range of 120/80-159/99 on two different occasions, daily sodium intake of over 2300 mg over a week time, A1C between 5.6-8.9, LDL between greater than 100, HDL of less than 40, being a smoker or having a BMI of greater than 25. The intervention pathway can also be selected when multiple measurements indicate a health condition. For example, the intervention pathway can be selected if, as indicated by the first patient health data 1008 and second patient health data 1010, the weight of the patient 1002 has increased by two pounds in a 24 hour period or five pounds in one week, and/or the blood pressure is 143-170/80-100, and/or pulse 100-120, and/or shortness of breath at activity, and/or mild swelling, and/or no cough, and no chest pain and no trouble lying flat (e.g., readings that are slightly deviated from normal ranges).

In some examples, the intervention pathway can be selected if the first patient health data 1008 indicates a predisposition for a health condition, and the second patient health data 1010 indicates that health measurements are trending towards a health condition. For example, even if blood pressure readings in the second patient health data 1010 are slightly below 120/80, the intervention pathway can still be selected if the first patient health data 1008 indicates that the patient 1002 has a genetic predisposition towards high blood pressure. In some examples, the intervention pathway can be selected if current blood pressure readings (even if in normal range) in the second patient health data 1010 and historical blood pressure readings in the first patient health data 1008 indicate an overall trend upward towards pre-hypertension.

The escalation pathway is selected when the first patient health data 1008 and the second patient health data 1010 indicate that the patient 1002 has an exigent health condition that does require immediate medical attention. The escalation pathway can be characterized by having a blood pressure greater than 160/100 repeatedly for more than two days, arrhythmia of some type detected by an IoT device such as the smart watch 1024 or having an A1C greater than 9. The escalation pathway can be selected based on several factors as well. For example, the escalation pathway can be selected if, as indicated by the first patient health data 1008 and the second patient health data 1010, the weight of the patient 1002 has increased by three pounds in 24 hours, the blood pressure is over 170/100, shortness of breath at rest, moderate to severe leg swelling, persistent dry cough, chest pain, trouble sleeping, and/or cannot life flat with two or less pillows or limited activity. For example, the first patient health data 1008 can store historical weights of the patient 1002 while the second patient health data 1010 can store a current weight of the patient 1002. A comparison of the current weight to the historical weights can indicate whether the patient has weight change (e.g., greater than three pounds).

FIG. 10B illustrates a table 1044 that shows conditions that can cause the RTHMRP 1006 to select between the stable, intervention, and escalation pathways. In some examples, the characteristics to determine to apply the stable, intervention or escalation pathway changes based on whether the patient is in a diagnosed or pre-diagnosed state. For example, in a diagnosed state the stable pathway can be characterized by blood pressure of less than 130/80, pulse less than 100, no symptoms, activity level normal, sodium intake less than two grams. The intervention pathway can be characterized by a weight increasing by two pounds in 24 hour period or 5 pounds in one week, blood pressure of 143-170/80-100, pulse rate of 100-120, shortness of breath at activity, and/or mild leg swelling with no cough, no chest pain and no trouble lying flat.

Thus, the RTHMRP 1006 analyzes the first health indication 1012a and the N health indication 1012n to determine whether the first health indication 1012a and the N health indication 1012n indicate a same health condition (e.g., high blood pressure) and/or a specific behavior, and if so, whether the health condition is exigent. Based on as much, the RTHMRP 1006 sets the action pathway 1032 as the stable pathway, the intervention pathway, or the escalation pathway. In this example, the first health indication 1012a indicates Stage I hypertension and the N health indication 1012n indicates a genetic predisposition to hypertension. Based on as much, the action pathway 1032 is set to the intervention pathway. In some cases, a state I hypertension diagnosis cannot trigger an intervention pathway if the patient 1002 did not have genetic predisposition to hypertension.

The RTHMRP 1006 can then generate a first action plan 1040 (e.g., can be a predefined plan that follows clinical standards or is generated on the fly by artificial intelligence) based on the action pathway 1032, and based on a level of urgency indicated in the action pathway 1032. For example, if the action pathway 1032 is the stable pathway, a low level of urgency is indicated and the first action plan 1040 can include periodic health measurements of the patient 1002, a notification of any observed health trends (e.g., increasing blood pressure but not yet in a hypertension group), health measurements, and a reward to the patient 1002. When the action pathway 1032 is the intervention pathway, a medium level of urgency is indicated and the first action plan 1040 includes one or more of a notification of a health condition or a potential health condition that is developing, health-related training, a live and/or online medical consultation, a healthcare provider visit, requests to continue to monitor health and provide updated health measurements, etc. In some examples, the first action plan 1040 can be immediate temporal next steps for the patient 1002 followed by a wait state for more information or for a timed event. When the action pathway 1032 is the escalation pathway, a high level of urgency is indicated and the first action plan 1040 includes an attempt to urgently connect the patient to a healthcare provider through the healthcare provider device 1030, an urgent notification to one or more of a healthcare provider via the healthcare provider device 1030 and the patient 1002 via the patient device 1028 that the patient 1002 is in a diseased state associated with the first and N health indications 1012a, 1012n, or an urgent deployment of emergency personnel to the patient 1002.

In this example, the action pathway 1032 is the intervention pathway. Therefore, the first action plan 1040 includes one or more of a notification of a health condition or a potential health condition that is developing, health-related training, a live and/or online medical consultation, a healthcare provider visit, requests to continue to monitor health and provide updated health measurements, etc. The exact nature of the first action plan 1040 can be set based on the particular health condition associated with the first health indication 1012a and N health indication 1012n. For example, when the health condition is hypertension, the healthcare providers can have an in-depth experience with hypertension, and the patient 1002 can be provided with guidance to lower blood pressure (e.g., reduce sodium, exercise, sleep regularly, etc.). Thus, the first action plan 1040 can be determined based on the health condition associated with the first health indication 1012a and N health indication 1012n.

The RTHMRP 1006 further includes a machine learning prediction model 1038. The machine learning prediction model 1038 can be trained based on historical data from the historical database 1036 associated with a plurality of patients. The historical database 1036 can include a plurality of action plans that are stored in association with the outcome data (e.g., whether each of the action plans resulted in prevention or reduction of a health condition). The machine learning prediction model 1038 is trained to predict whether an action plan in the historical data is associated with a successful outcome based on outcome data of the historical data. The machine learning prediction model 1038 can generate the first action plan 1040.

In some examples, the machine learning prediction model 1038 generates the first action plan 1040 prior to analysis of the first and second patient health data 1008, 1010. The first action plan 1040 is selected in response to the action pathway 1032 and the health condition associated with the first and N health indications 1012a, 1012n. In such embodiments, the machine learning prediction model 1038 generates a plurality of action plans. In other embodiments, the first action plan 1040 can be generated on the fly and based on specific health factors (e.g., the condition, age, demographic, geography, comorbidities, etc.) associated with the patient 1002 and as identified from the first patient health data 1008. In some examples, the machine learning prediction model 1038 does not generate the first action plan 1040, and rather the RTHMRP 1006 determines the first action plan 1040 based on predefined treatment pathways that may have been derived from machine learning type analytics.

In some examples, the machine learning prediction model 1038 can associate the first health indication 1012a to the first patient health data 1008 and the second patient health data 1010 to the N health indication 1012n, generate the action pathway 1032 and the first action plan 1040. For example, the machine learning prediction model 1038 is trained based on historical data from the historical database 1036, where the historical data is associated with a plurality of patients. The machine learning prediction model 1038 is trained based on whether health measurements (e.g., patient data) in the historical data is associated with health indications in the historical database 1036. The machine learning prediction model 1038 therefore can determine whether health measurements are associated with a health indication during inference. In some examples, the machine learning prediction model 1038 includes one or more neural networks.

The RTHMRP 1006 can gather the historical data over a period of time, and store the historical data into the historical database 1036. For example, the RTHMRP 1006 can track interactions, action pathways, and outcomes of other patients and store as much in the historical database 1036. In some embodiments, the historical database 1036 can be a blockchain storage that is encrypted and where the data is stripped of personal identifying information to preserve patient anonymity and enhance security.

The RTHMRP 1006 can generate an outreach event to provide the first action plan 1040 and other relevant information to one or more of the patient device 1028 and/or the healthcare provider device 1030. The patient device 1028 can display a graphical patient interface that notifies the patient 1002 of the health condition and the first action plan 1040. In some examples, the patient device 1028 can implement aspects of the first action plan 1040 such as scheduling a live and/or online consultation with a health professional and periodically reminding the patient 1002 to track and maintain a low sodium level, asking for inputs verifying that the patient 1002 is complying with the first action plan 1040, etc.

In some examples, the RTHMRP 1006 tracks whether the patient 1002 is complying with the first action plan 1040. For example, the patient 1002 can provide inputs (e.g., sodium counts, estimation of aerobic activity, etc.) into the patient device 1028 to signal compliance with the first action plan 1040. The patient device 1028 provides the inputs to the RTHMRP 1006.

The RTHMRP 1006 also includes a rewards engine 1042. The rewards engine 1042 can generate rewards based on compliance of the patient 1002 with the first action plan 1040. For example, the first action plan 1040 can include a request for periodic blood pressure readings at certain times. If the patient 1002 diligently monitors blood pressure of the patient 1002 with the blood pressure cuff 1026 and provides the pressure readings to the RTHMRP 1006 at the certain times, the rewards engine 1042 can determine that the patient is complying with the first action plan 1040 and therefore generate/provision a reward (e.g., monetary compensation, congratulations message, merchandise, etc.) for the patient 1002. In contrast, if the patient 1002 does not provide the pressure readings to the RTHMRP 1006 at the certain times, then the rewards engine 1042 can determine that the patient 1002 is not complying with the first action plan 1040 and generate a penalty based on the non-compliance of the patient 1002.

In some embodiments as explained further below, the RTHMRP 1006 can adjust or replace the first action plan 1040 in response to an identification that the patient 1002 is not complying with the first action plan 1040. For example, the machine learning prediction model 1038 can include a generative artificial intelligence model that generates new action plans based on actions and/or an analysis of the patient 1002. For example, data of the patient 1002 can be retrieved from the patient database 1034 and provided to the generative artificial intelligence model as inputs. Furthermore, the noncompliance of the patient 1002 can be provided to the generative artificial intelligence model as inputs. The generative artificial intelligence model can generate a new plan that can better address the patient's 1002 needs while also being adjusted to the patient's 1002 personality. For example, the data of the patient 1002 can be indicative of personality traits and habits of the patient 1002 (e.g., enjoys exercising, dislikes jogging, enjoys biking, etc.). The generative artificial intelligence model can adjust plans based on the identified personality traits and habits to provide a focused and engaging plan tailored to the patient 1002. Doing so results in better compliance, resulting in reduced resources to treat the patient 1002 and better outcomes.

In some examples, the RTHMRP 1006 can also include a chat interface that is available to the patient 1002. For example, the chat interface can be presented on a graphical user interface of the patient device 1028. The chat interface can operate with a generative artificial intelligence model operating on the RTHMRP 1006. The generative artificial intelligence model can provide instructions, guidance, answers (e.g., doctor recommendations, exercise advice, etc.) to the patient 1002 in an interactive format.

In some examples, activity information of the patient 1002 can be entered manually and/or tracked automatically by the smart watch 1024. The activity information can be stored and accumulated (e.g., track activity over a week) in the patient database 1034. The RTHMRP 1006 can identify the activity of the patient 1002 based on the activity information stored in the patient database 1034. The RTHMRP 1006 can determine whether the activity corresponds to an expected action associated with the first action plan 1040. The RTHMRP 1006 can determine whether the patient 1002 is following the first action plan 1040 based on whether the activity corresponds to the expected action. If the patient 1002 is following the first action plan 1040, the RTHMRP 1006 can maintain the first action plan 1040, generate a reward, and provide the reward to the patient 1002. If the patient 1002 is not in compliance with the first action plan 1040, the RTHMRP 1006 can generate a penalty for the patient 1002, adjust the first action plan 1040 to generate a second action plan, and transmit a command to the patient device 1028 to implement the second action plan.

Thus, the aforementioned embodiments generate a first action plan 1040 based on granular data retrieval and analysis. The first action plan 1040 can be customized based on the specific characteristics of the patient 1002. Moreover, embodiments can execute in real-time to reduce latency to diagnose and treat the patient 1002. Furthermore, embodiments can reduce the level of resources needed to effectively diagnose and treat the patient 1002.

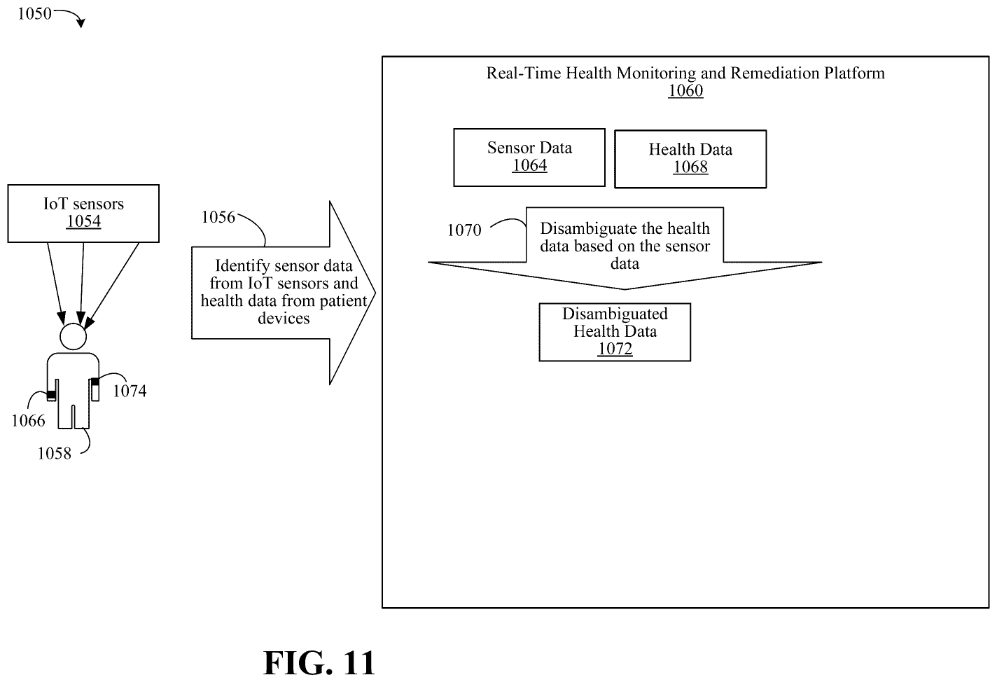

Turning now to FIG. 11, a noise reduction process 1050 is illustrated. The noise reduction process 1050 can be implemented in conjunction with any of the embodiments described herein, including the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), neural network 902 (FIG. 9) and/or the health monitoring, identification and treatment process 1000 (FIGS. 10A and 10B).

In particular, a patient 1058 wears a plurality of devices (e.g., a blood pressure cuff, smart watch, fitness tracker, etc.) including a blood pressure cuff 1074 and a smart watch 1066 to measure health characteristics of the patient 1058. As noted above, certain factors can interfere with retrieving accurate measurements (e.g., time of day, stress level, environmental conditions such as heat, cold, etc.). Internet-of-things (IoT) sensors 1054 generate sensor readings (e.g., motion sensor, temperature readings, environmental conditions, audio sensor, proximity sensors, infrared sensor, touch sensor, color sensor, humidity sensor, etc.) associated with the patient 1058 to weight obtained health measurements of the patient 1058 and reduce noisy (inaccurate) measurements. For example, the noise reduction process 1050 identifies sensor data 1064 from the IoT sensors 1054 and health data 1068 from the patient devices 1056. A RTHMRP 1060 receives and stores the health data 1068 and the sensor data 1064.

The RTHMRP 1060 disambiguates the health data 1068 (e.g., blood pressure readings pulse rate, etc.) based on the sensor data 1064, 1070. For example, suppose that the health data 1068 is a blood pressure measurement that is in the hypertension group. Further suppose the sensor data 1064 indicates that the patient 1058 coughed as the blood pressure measurement was taken by the blood pressure cuff 1074. The RTHMRP 1060 can reduce the weight of the blood pressure measurement (e.g., set weight to a low value or 0) since the cough could cause the blood pressure to temporarily spike causing an unusually high blood pressure measurement. The disambiguated health data 1072 is the weighted blood pressure measurement in this example.

In another example, suppose that the smart watch 1066 detects a high pulse rate and provides as much as part of the health data 1068. Further suppose that the sensor data 1064 indicates that the patient 1058 was exercising when the high pulse rate was measured. The RTHMRP 1060 can reduce the weight of the high pulse rate (e.g., set weight to a low value or 0) since the high pulse rate is caused by exercise causing a temporary pule rate. The disambiguated health data 1072 is the weighted pulse rate in this example.

While not illustrated, the RTHMRP 1060 can discard the disambiguated health data 1072 altogether if the weight is below a threshold. In some examples, if the disambiguated health data 1072 is not discarded, the RTHMRP 1060 can associate the disambiguated health data 1072 with a health indication, generate an action pathway and generate an action plan as described with respect to the RTHMRP 1006 of FIG. 10A. It is worthwhile to note that the RTHMRP 1006 (FIG. 1) can readily execute any of the functions described with respect to the RTHMRP 1060 and in conjunction with the operations of the RTHMRP 1006.

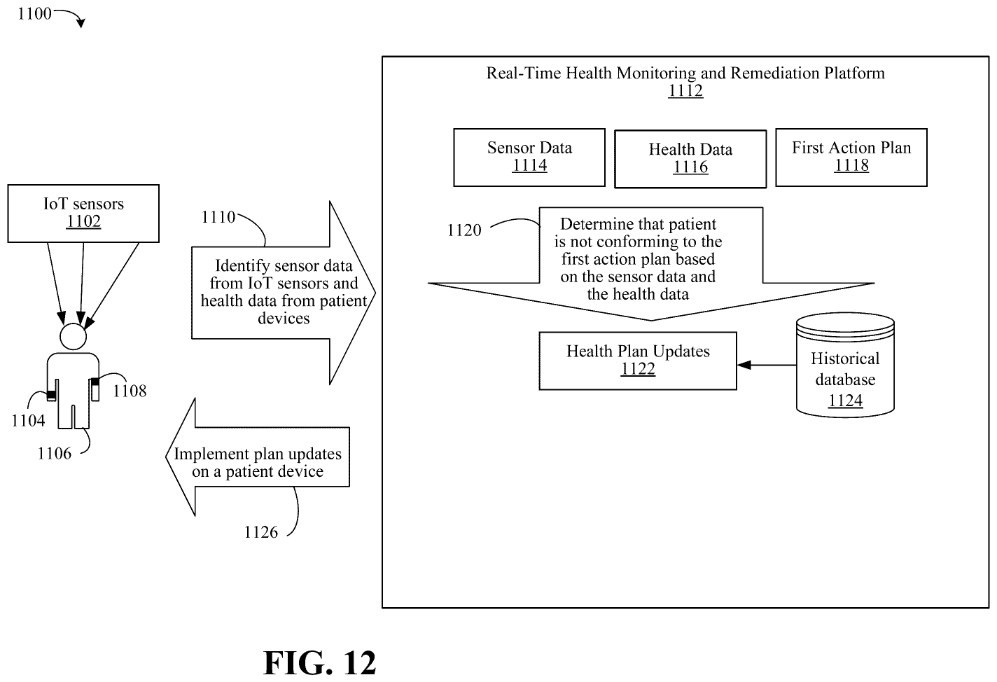

FIG. 12 illustrates a health plan compliance process 1100. The health plan compliance process 1100 can be implemented in conjunction with any of the embodiments described herein, including the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), neural network 902 (FIG. 9), the health monitoring, identification and treatment process 1000 (FIGS. 10A and 10B), and/or noise reduction process 1050 (FIG. 11).

A RTHMRP 1112 is illustrated. The RTHMRP 1112 previously determined that a first action plan 1118 should be followed by patient 1106. The RTHMRP 1112 periodically determines if the patient 1106 is in compliance with the first action plan 1118.

For example, the patient 1106 is wearing a plurality of wearable patient devices 1104, 1108 that measure health data 1116 of the patient 1106. IoT sensors 1102 can also sense conditions related to the patient such as movement of the patient 1106, environment of the patient 1106, temperature of the patient 1106, etc. The process 1100 identifies sensor data 1114 from the IoT sensors 1102 and health data 1116 from the patient devices 1104, 1110.

The RTHMRP 1112 determines whether the patient 1106 is conforming to the first action plan 1118 based on the sensor data 1114 and the health data 1116. For example, if the first action plan 1118 indicates that the patient 1106 is to exercise for 150 minutes per week, the RTHMRP 1112 can verify that the patient 1106 is indeed meeting the mandated exercise time. That is, exercise amounts can be recorded by IoT sensors 1102 (e.g., motion detectors to detect exercise related movements) as the sensor data 1114 and/or the patient devices 1104, 1108 (e.g., smartwatch that records when heart rate reaches exercise related levels, and/or exercise related movements) as health data 1116. In some examples, other patient devices (e.g., computing devices) can also provide data indicating whether the patient is in compliance with the first action plan 1118.

The RTHMRP 1112 identifies if the patient 1106 is in compliance with the first action plan 1118. In this example, the RTHMRP 1112 determines that the patient 1106 is not conforming to the first action plan 1118 based on the sensor data 1114 and the health data 1116, 1120. In response to the patient 1106 not conforming to the first action plan 1118, the RTHMRP 1112 generates health plan updates 1122 to cause greater compliance in the patient 1106. For example, the RTHMRP 1112 can generate a first metric for the first action plan 1118 based on whether the patient 1106 is in compliance with the first action plan 1118. The first metric is a probability that the patient 1106 will comply with the first action plan 1118. The probability can be determined based on the user compliance/non-compliance with the first action plan 1118.

The RTHMRP 1112 can generate a second metric for a second action plan, embodied as the health plan updates 1122, based on historical data retrieved from a historical database 1124. The historical data is associated with a plurality of patients. For example, the RTHMRP 1112 can detect whether different action plans were effective for treating an underlying health condition of the patient 1106. The RTHMRP 1112 can select the action plan from the different action plans, excluding the first action plan 1118, with the highest probability of compliance for the patient 1106. The highest probability can be the second metric.

The RTHMRP 1112 can cease implementation of the first action plan 1118 and implement the second action plan based on a comparison of the first metric and the second metric. That is, the probability of the second metric is greater than the probability of the first metric, and thus the first action plan 1118 is replaced with the second action plan. The second action plan is stored as the health plan updates 1122. The process 1100 then implements plan updates (e.g., notify the patient of the second action plan) on a patient device 1126.

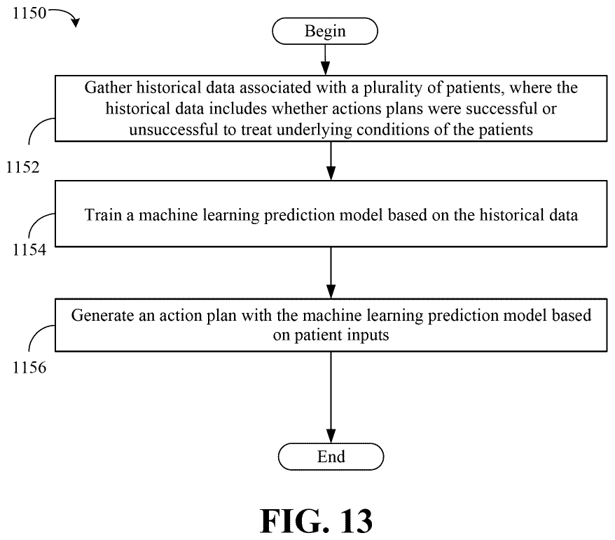

FIG. 13 shows a method 1150 of generating an action plan. The method 1150 can generally be implemented in conjunction with any of the embodiments described herein, for example the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), and neural network 902 (FIG. 9), the health monitoring, identification and treatment process 1000 (FIGS. 10A and 10B), the health monitoring, identification and treatment process 1000 (FIG. 10A) noise reduction process 1050 (FIG. 11), and/or health plan compliance process 1100 (FIG. 12). In an embodiment, the method 1150 is implemented in logic instructions (e.g., software), configurable logic, fixed-functionality hardware logic, computer readable instructions stored on at least one non-transitory computer readable storage medium that are executable to implement method 1150, circuitry, etc., or any combination thereof.

Illustrated processing block 1152 gathers historical data associated with a plurality of users, where the historical data includes whether actions plans were successful or unsuccessful to treat underlying conditions of the patients. Illustrated processing block 1154 trains a machine learning prediction model based on the historical data. For example, if machine learning prediction model is a neural network, processing block 1154 can include generating a loss based on the historical data and updating parameters of the neural network based on the loss. Illustrated processing block 1156 generates an action plan with the machine learning prediction model based on patient inputs. For example, the patient inputs can include sensor data related to the patient, health related readings of the patient, patient data (e.g., family history, previous readings, etc.) of the patient, which are then processed by the neural network to select an appropriate action plan.

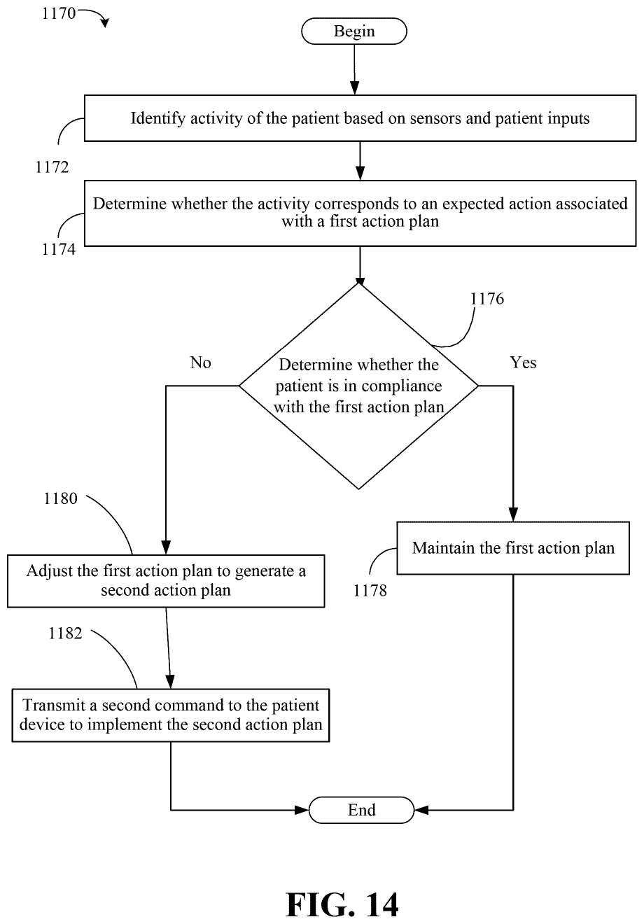

FIG. 14 shows a method 1170 of improving compliance with an action plan. The method 1170 can generally be implemented in conjunction with any of the embodiments described herein, for example the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), neural network 902 (FIG. 9), the health monitoring, identification and treatment process 1000 (FIGS. 10A and 10B), noise reduction process 1050 (FIG. 11), health plan compliance process 1100 (FIG. 12) and/or method 1150 (FIG. 13). In an embodiment, the method 1170 is implemented in logic instructions (e.g., software), configurable logic, fixed-functionality hardware logic, computer readable instructions stored on at least one non-transitory computer readable storage medium that are executable to implement method 1170, circuitry, etc., or any combination thereof.

Illustrated processing block 1172 identifies activity of the patient based on sensors and patient inputs (e.g., manual inputs by the patient into a computing device). Illustrated processing block 1174 determines whether the activity corresponds to an expected action associated with a first action plan (e.g., executes an action listed in the first action plan). Illustrated processing block 1176 determines whether the patient is in compliance with the first action plan, for example based on whether the activity corresponds to the expected action. If so, illustrated processing block 1178 maintains the first action plan. Otherwise, illustrated processing block 1180 adjusts the first action plan to generate a second action plan. Processing block 1180 can include adjusting features of the first action plan, such as suggested exercise types, duration, food suggestions etc. Illustrated processing block 1182 transmits a second command to the patient device to implement the second action plan.

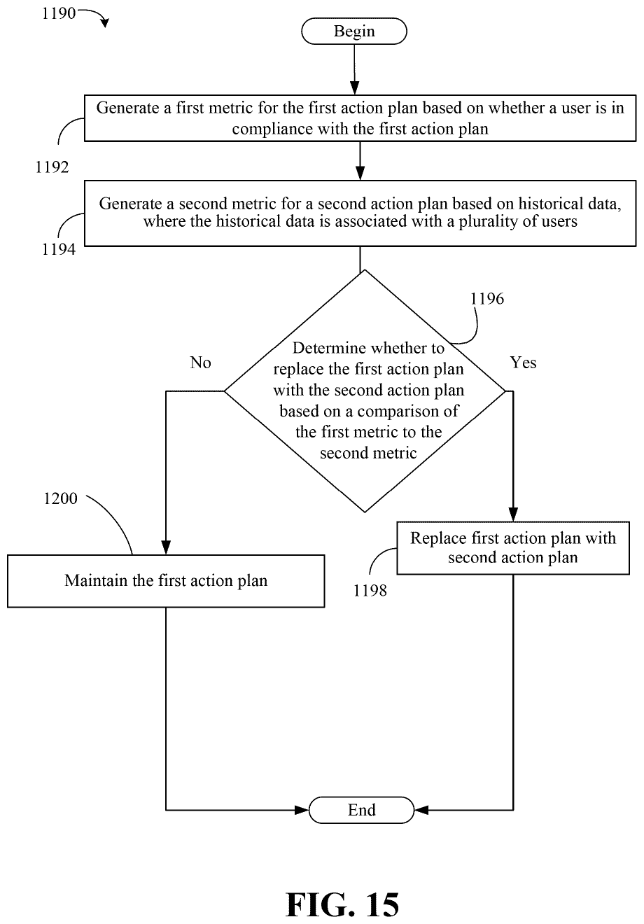

FIG. 15 shows a method 1190 of switching action plans. The method 1190 can generally be implemented in conjunction with any of the embodiments described herein, for example the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), and neural network 902 (FIG. 9) and the health monitoring, identification and treatment process 1000 (FIGS. 10A and 10B), noise reduction process 1050 (FIG. 11), health plan compliance process 1100 (FIG. 12), method 1150 (FIG. 13) and/or method 1170 (FIG. 14). In an embodiment, the method 1190 is implemented in logic instructions (e.g., software), configurable logic, fixed-functionality hardware logic, computer readable instructions stored on at least one non-transitory computer readable storage medium that are executable to implement method 1190, circuitry, etc., or any combination thereof.

Illustrated processing block 1192 generates a first metric for a first action plan based on whether a user is in compliance with the first action plan. Illustrated processing block 1194 generates a second metric for a second action plan based on historical data, where the historical data is associated with a plurality of users. Illustrated processing block 1196 determines whether to replace the first action plan with the second action plan based on a comparison of the first metric (e.g., a probability of compliance with the first action plan based on whether user actions are part of the first action plan) to the second metric (e.g., a probability of compliance with the second action plan determined based on the historical data). If it is determined that the first action plan is to be replaced (e.g., second metric is greater than first metric), illustrated processing block 1198 replaces the first action plan with the second action plan. If it is determined that the first action plan (e.g., first metric is equal to or greater than second metric) is to be maintained, then illustrated processing block 1200 maintains the first action plan.

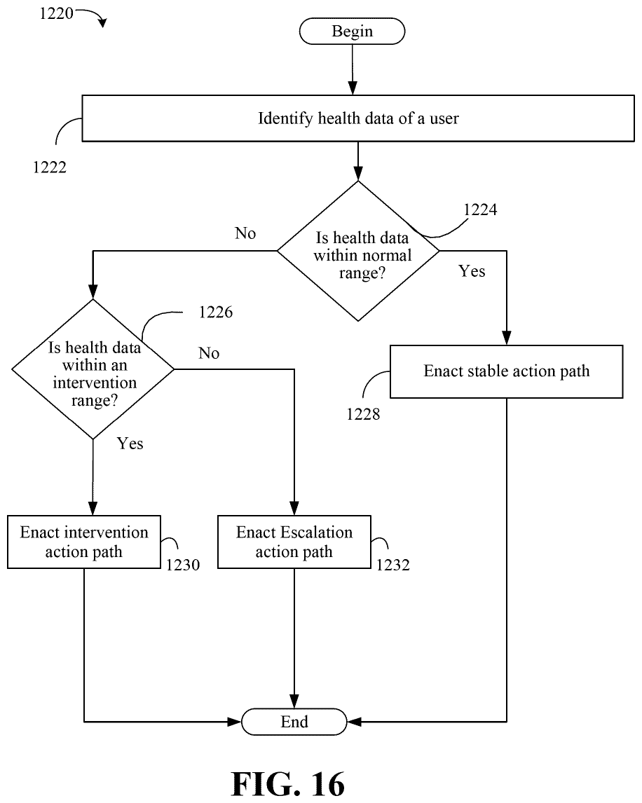

FIG. 16 shows a method 1220 of selecting action pathways. The method 1220 can generally be implemented in conjunction with any of the embodiments described herein, for example the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), neural network 902 (FIG. 9) and the health monitoring, identification and treatment process 1000 (FIGS. 10A and 10B), noise reduction process 1050 (FIG. 11), health plan compliance process 1100 (FIG. 12), method 1150 (FIG. 13), method 1170 (FIG. 14) and/or method 1190 (FIG. 15). In an embodiment, the method 1220 is implemented in logic instructions (e.g., software), configurable logic, fixed-functionality hardware logic, computer readable instructions stored on at least one non-transitory computer readable storage medium that are executable to implement method 1220, circuitry, etc., or any combination thereof.

Illustrated processing block 1222 identifies health data of a user. Illustrated processing block 1224 determines if the health data is within a normal range. If not, illustrated processing block 1226 determines if the health data is within an intervention range (e.g., health data is slightly deviated from normal ranges). If not, illustrated processing block 1232 enacts an escalation action pathway. If the health data is within intervention range, illustrated processing block 1230 enacts an intervention action pathway. If processing block 1224 determines that the health data is within the normal range, illustrated processing block 1228 enacts a stable action pathway.

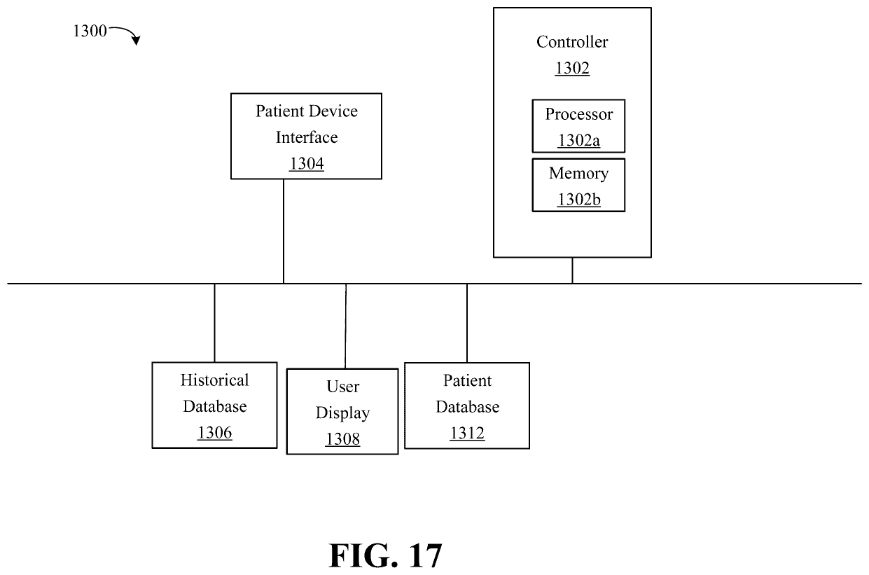

FIG. 17 shows a more detailed example of a computing system 1300 to diagnose a health condition, select an action plan and track compliance with the action plan. The illustrated computing system 1300 can be readily included in for example the health monitoring system 100 (FIG. 1), database 152 (FIG. 2), outreach event selection system 156 (FIG. 3), user interfaces 400 (FIG. 4) and 500 (FIG. 5), process 600 (FIG. 6), software architecture 706 (FIG. 7), machine 800 (FIG. 8), neural network 902 (FIG. 9) and the health monitoring, identification and treatment process 1000 (FIGS. 10A and 10B), noise reduction process 1050 (FIG. 11), health plan compliance process 1100 (FIG. 12), method 1150 (FIG. 13), method 1170 (FIG. 14), method 1190 (FIG. 15) and/or method 1220 (FIG. 16).

In the illustrated example, the computing system 1300 can include a patient device interface 1304 that can communicate with external devices (e.g., mobile devices, computers, smart watches, IoT devices, weigh scales, blood pressure cuff, etc.) to receive health measurements, environmental measurements and other data associated with the patient. The computing system 1300 also includes a user display 1308 (e.g., audio and/or visual interface) to display action plans and receive instructions from a user (e.g., an operator). The instructions can indicate an adjustment to an action plan. A historical database 1306 stores action plan data and outcomes associated with a plurality of patients, and a patient database 1312 stores information specific to the patient such as EMRs, family history information, previous health measurements, etc.

A controller 1302 can identify a patient, select first health data associated with the patient from a patient database, where the first health data includes a first health measurement of the patient, determine that the first health measurement is associated with a first health indication, where the first health indication is associated with a health condition, receive second health data in real-time from a first computing device associated with the patient, where the second health data is associated with the patient, determine that a second health measurement of the second health data is associated with a second health indication, select an action pathway from a first pathway, a second pathway and a third pathway based on the first and second health measurements being associated with the first and second health indications, automatically select a first action plan based on the action pathway. The first action plan includes a process flow to one or more of remediate the health condition and prevent an occurrence of the health condition in the patient, and the controller 1302 transmits a first outreach event to a second computing device of the patient over a computer network to implement the first action plan in real-time. The controller 1302 includes a processor 1302a (e.g., embedded controller, central processing unit/CPU) and a memory 1302b (e.g., non-volatile memory/NVM and/or volatile memory) containing a set of instructions, which when executed by the processor 1302a, cause the controller 1302 to generate an action plan based on data received from the patient device interface 1304, historical database 1306 and patient database 1312.

The computing systems described herein can add the real time monitored data to a longitudinal patient record (LPR), which can include a comprehensive medical profile of the patient sourced from numerous care providers, pharmacies, hospitals, third-party medical data vendors, the patient data, and the claims data, etc. The LPR can be a machine readable file that stores data relating to the individual's health. The manager device can create the LPR by compiling or aggregating received medical data (e.g., files) from numerous channels, including data already stored in the storage device, as will be described in greater detail below. The LPR can include patient demographics, encounters with care providers, patient vital signs gathered by care providers, medications prescribed to the patient, immunizations that the patient received, procedures performed on the patient, test results from tests performed on the patient, health goals set by the patient or the patient's care provider, care plans or medical treatment plans created by care providers for the patients, diagnoses or other health conditions of the patient, and social determinants of health. The LPR can further include data contributed by a user. For example, data contributed by a user can include information provided from a wearable or other device capable of providing health information. In some embodiments, the user may manually provide health information from an app or a website. The manager device can create the LPR in response to various external trigger messages, as will be described in more detail below. Additionally, the LPR can include a completeness score generated by the manager device or another device connected to the network. The completeness score can provide a numerical value indicative of the medical data's comprehensiveness and accuracy, which can provide added confidence to care providers seeking to use the LPR to learn about the patient before a patient arrives for medical care or to guide how medical care is to be administered. For example, an unconscious patient cannot answer medical history questions, so the doctor may rely on an LPR having a high completeness score to make informed medical decisions about the unconscious patient, which may save a patient's life (e.g., the doctor avoids certain medications identified as causing an allergy to the unconscious patient during the course of care). A completeness score for an individual can be based on the particular care path assigned to the patient's LPR. In an example embodiment, the completeness score can be weighted to reflect a particular procedure assigned to patient. In some circumstances a first completeness score relates to the overall health records of the patient and a second, specific procedure completeness score is assigned for a particular procedure/medical visit to be undertaken by the patient.

In one particular example, the manager device can create the LPR by aggregating or compiling patient data, claims data, and external data received from the health network utility in response to receiving an accept/discharge/transfer (ADT) message from a care provider on the network. In some embodiments, the ADT message comprises Health Level Seven (HL7) data transmitted on the network, and the ADT message can represent data associated with numerous medical events including admission of a patient to a care facility, discharging a patient from a care facility, changing an inpatient designation to an outpatient designation or vice versa, changing a patient ID, transferring a patient, or any other ADT message defined by HL7 or other protocol. The present system can be triggered to update the LPR upon an update to the patient record within a provider device or between provider devices. When different devices exchange patient related data, e.g., interface with each other, to send or receive new/updated patient data, that data is sent to the LPR.

The virtual charts comprise materialized views of data included in one or more than one LPRs. Said differently, each virtual chart can present data included in one or more than one LPRs and present the data in a specialized way according to preferences of a person or device that requested the virtual chart. For example, a first doctor may be an allergist, and the allergist may not need all medical data stored in the LPR, such as data submitted by an orthopedist, and so the virtual chart presented to the allergist may only contain some of the LPR data that is relevant to treating a patient's allergies. In another example, the manager device may generate a first virtual chart with medical data presented in a table form for a first data consumer (e.g., a first doctor), and the manager device may generate a second virtual chart with the same medical data presented as a graph for a second data consumer (e.g., a second doctor). The manager device may receive preferences from a data consumer, such as one of the provider devices or the user device, and generate a virtual chart in response to receiving those parameters or in response to other query information provided at the time the virtual chart was requested. In another embodiment, the preferences may be previously set and stored in the storage device. The virtual charts can also change in format depending on the time requested because medical data may have changed between a first virtual chart request and a second virtual chart request.

Example methods and systems for updating and curating data are described. More specifically, example methods and systems for receiving medical data from numerous sources or channels at defined trigger messages or events and curating the data at the defined trigger messages or events to create an LPR are described. Furthermore, example methods and systems for presenting updated and curated data according to user preferences is described. In the following description, for purposes of explanation, numerous specific details are set forth in order to provide a thorough understanding of example embodiments. It will be evident, however, to one of ordinary skill in the art that embodiments of the present disclosure may be practiced without these specific details.

Typically, medical data for an individual is scattered across numerous data storage locations controlled by various entities. For example, a hospital may store an EMR for an individual on a data storage device controlled by the hospital, and the hospital's EMR may store medical data related to an emergency room visit by the individual or surgery performed at the hospital on the individual. Meanwhile, a pharmacy may store prescription drug fulfillment history for the individual on a data storage device controlled by the pharmacy, and a primary care doctor may store vital sign information from yearly physicals administered to the individual on a data storage device controlled by the primary care physician's office. However, in this example, none of the hospital, the pharmacy, or the primary care physician has a complete medical record for the individual. In addition, medical data for the individual may be stale at one or more than one of the above-described entities. For example, the hospital may have vital sign data when a patient had a knee surgery operation in 2015, but the patient may have since developed a high blood pressure condition, rendering the hospital's blood pressure reading stale, outdated, and of little value.

Creating a single electronic health record for an individual reflecting all the medical events associated with the individual can be very difficult. To begin, a single entity would need to receive data from all care providers that provided care to the individual, and the single entity would need to convince every care provider to share this private medical data. Additionally, while medical data networks exist whereby entities, such as insurance companies or other care providers, can request information related to an individual, the information may not have a consistent format, may have incorrect, outdated, or conflicting information, or may simply be too much information for a single computer to manage. Said differently, gathering all EMRs associated with a single individual may not actually provide an accurate or complete health picture of the individual even using the existing networks and medical data sharing providers that currently exist. Thus, there is a need to curate all medical data associated with each individual in order to create a comprehensive and accurate EMR for individuals.

While some methods for curating data might exists, such curation methods are either use case based or constant. Constant medical data curation causes an incredible strain on data computing resources. Such constant medical data curation is costly because it requires frequent requests to either the provider devices or the health network utility, and requests to either of those entities may cost money to or impact the resources of the requester. In addition, even if a device associated with the requesting entity were able to make frequent, inexpensive or free requests for medical data, the processing power required to curate massive amounts of data would be unreasonably burdensome, particularly for a significantly large population of individuals, a population size that may be desirable to generate a large enough sample size to run certain analytic processes. For example, an insurance company may have millions of patients, and constantly curating medical data for millions of patients may not be feasible, efficient, or possible for even modern computing. Use case curation, on the other hand, may not generate an accurate snapshot of an individual's health because the curation is not run at appropriate times or might become stale very quickly. In either situation, better data curation methods are needed in the medical field (or any other field, such as government benefits, financial records, credit reports, etc.)

Finally, even if an entity were able to efficiently curate medical data from numerous sources or channels, the data must still be accurate enough to be useful for data consumers, such as care providers or insurance companies seeking to manage patient risk. That is, simply because an entity can combine medical data from multiple sources does not mean that the data is accurate or useful to other data consumers (e.g., doctors, care providers, pharmacies, etc.). Thus, the data must be curated in an effective manner so that the data reflects the most updated, most accurate, and most complete representation of an individual's health. If medical data created from multiple sources were not curated in this manner, the data consumers may not trust the data or may not find it useful for providing care, and the data consumers may try to acquire the medical data through other means (e.g., asking the patient to fill out numerous forms, generating the data themselves, etc.).

At the same time, any medical record must be complete to provide an accurate representation of the medical conditions and care, which requires management of both historical data and current state data. For example, an individual may have developed high blood pressure 10 years ago, but due to diet, exercise, lowered stress, medication, or all the above, the individual no longer suffers from high blood pressure. A complete medical record should store historical data because the individual was, at one time, diagnosed with high blood pressure, and knowing that medical history may be useful for some medical procedures or care. However, the medical record must also recognize the current state of the individual, whereby the individual's vital signs no longer exhibit high blood pressure. Thus, to generate a complete and accurate medical record, a computer would differentiate, categorize, and present data meaningfully, depending on context or preferences, so that a care provider or other data consumer can understand both the patient's medical history and current status. Moreover, data as presented should make sense to the recipient. For example, a patient may not understand medical data in the form of test readings and complicated medical terminology, whereas a doctor would. More granularly, a first doctor may not find all medical data associated with an individual relevant to his, her, or their practice, and so, irrelevant information should not be presented to every care provider. So, data presentation should account for the audience in how it is presented so that the data provided best communicates medical condition to a recipient.

The systems and methods described herein address the above-described issues by curating data in response to meaningful trigger messages. The trigger messages may be related to medical health events, which generate messages on medical networks, such as HL7 messages or any other protocol. For example, the trigger messages can include ADT messages, insurance claim submissions, appointment scheduling, and insurance eligibility submissions. By curating data in response to trigger messages, the processing load on the manager device is reduced so that data curation is only performed at meaningful times. Moreover, the identified trigger messages listed above represent that an individual is about to undergo medical care, is currently undergoing medical care, or recently received medical care, meaning that the trigger represents a significant and meaningful time in the health history of the patient. Thus, curating the data at that point indicates that any request made in response to the trigger should result in a complete medical picture for the individual. In response to one of the trigger messages, the systems and methods described herein can receive data from numerous sources, compiling or aggregating the received data, and curate the data, thereby creating an LPR.

The present machine learning systems and methods described herein can run on the LPR, which may provide insights for generating models of healthcare across populations and for specific individuals to be assigned care paths.

This disclosure further relates to interactive digital personalized experience interfaces, and in particular for providing an interactive digital personalized experience interface for users to engage in various aspects of behavioral healthcare.

Healthcare management is increasingly becoming a complex and important aspect of daily life. As health aspects of conditions of an individual change over time, the individual typically engages in various healthcare treatments, procedures, therapies, and the like. Managing such aspects of the health of the individual can be burdensome, complex, and/or difficult and may result in noncompliance with healthcare treatments, procedures, therapies, and the like, which may provide less than optimal healthcare for the individual.

As described, healthcare management is increasingly becoming a complex and important aspect of daily life. As health aspects of conditions of an individual change over time, the individual typically engages in various healthcare treatments, procedures, therapies, and the like. Managing such aspects of the health of the individual can be burdensome, complex, and/or difficult and may result in noncompliance with healthcare treatments, procedures, therapies, and the like, which may provide less than optimal healthcare for the individual.

Accordingly, systems and methods, such as those described herein, configured to provide a personalized experience interface for managing aspects of the healthcare of an individual, may be desirable. In some embodiments, the systems and methods described herein may be configured to provide proprietary personalized care including one or more personalized experience components generated and/or selected based on enrollment criteria, insurance plan structure, care pathway considerations, additional care pathway considerations, and/or preferences stated or provided by a user. The systems and methods described herein may be configured to provide dynamic engagement components as a part of a checklist or wellness dashboard.

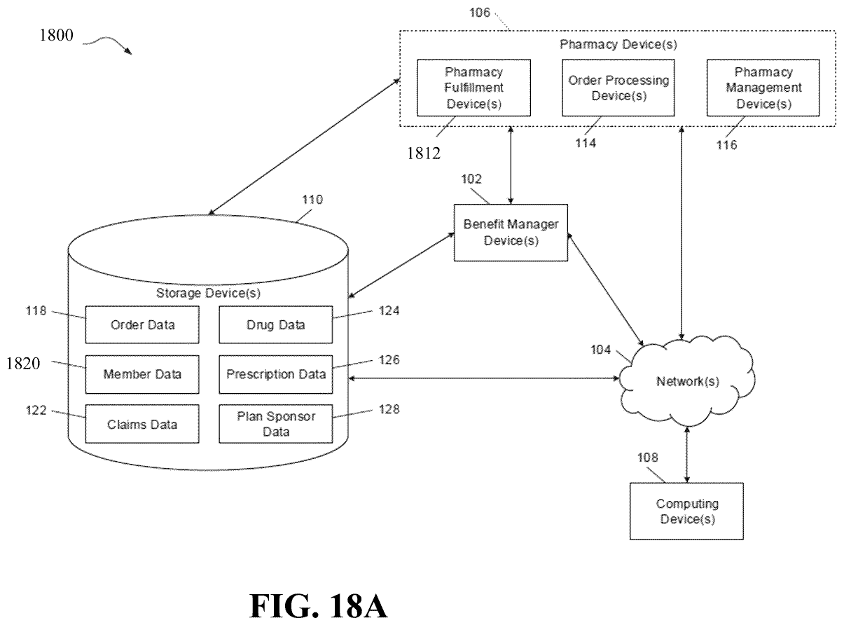

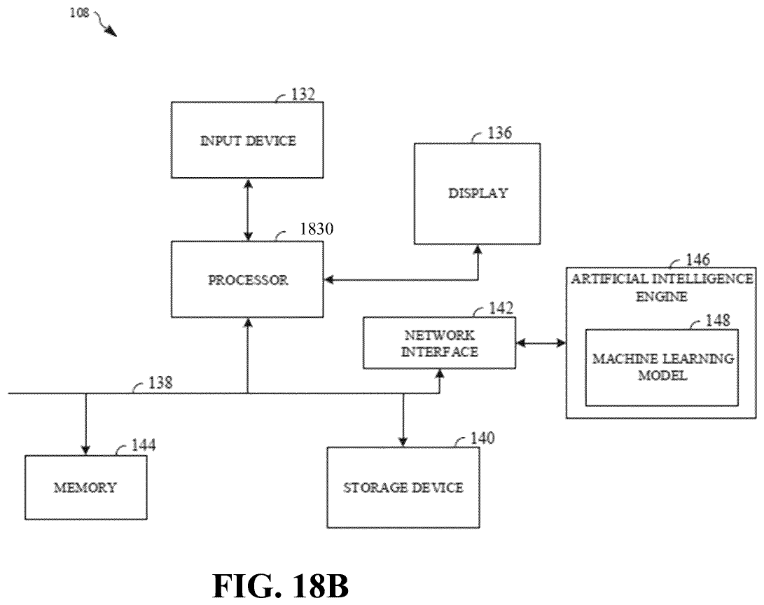

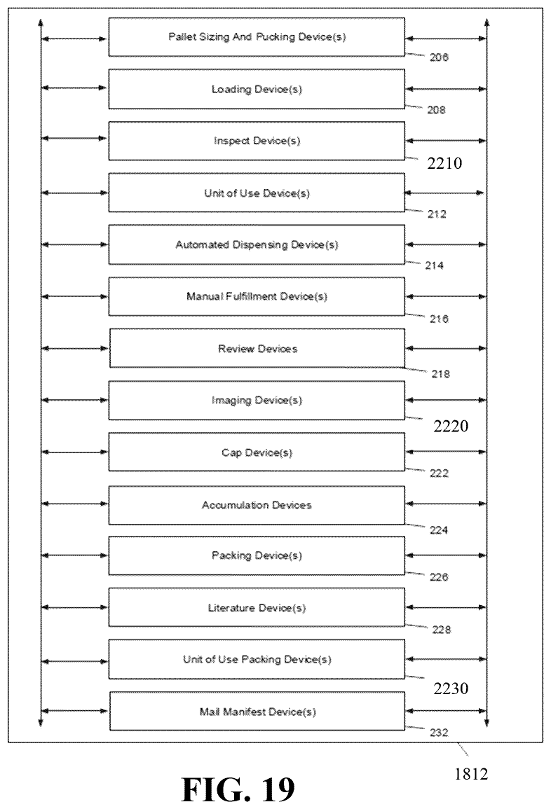

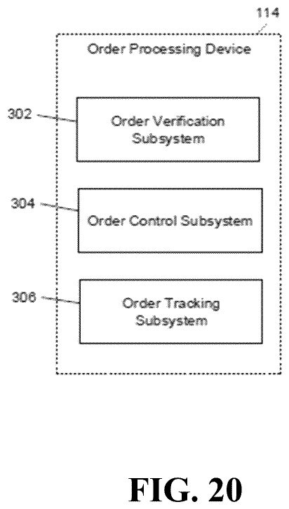

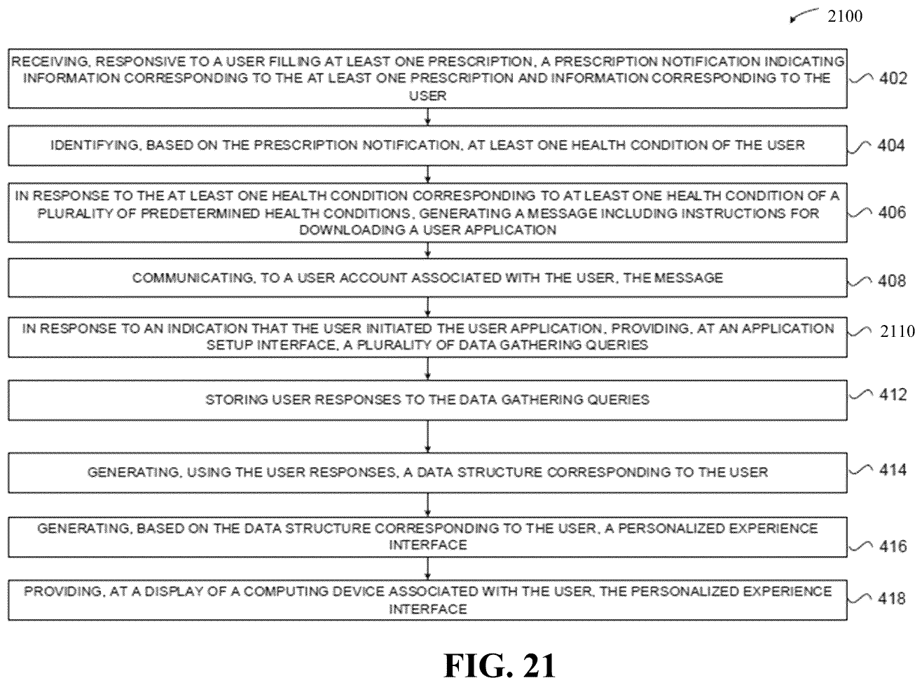

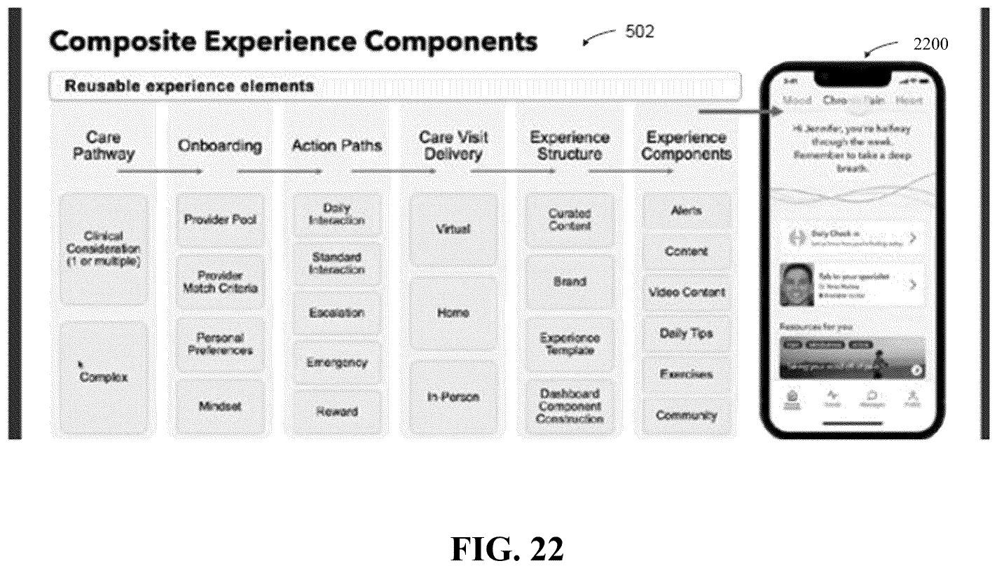

As is generally illustrated in FIG. 22, the systems and methods described herein may be configured to provide composed experience and composite customer experience, generally illustrated at 502. FIG. 22 is readily combinable with examples herein, for example health monitoring system 100 (FIG. 1), database 152 (FIG. 2), outreach event selection system 156 (FIG. 3), user interfaces 400 (FIG. 4), user interface 500 (FIG. 5), process 600 (FIG. 6), software architecture 706 (FIG. 7), 800 (FIG. 8), neural network 902 (FIG. 9), 1000 (FIG. 10A), table 1044 (FIG. 10B), noise reduction process 1050 (FIG. 11), health plan compliance process 1100 (FIG. 12), method 1150 (FIG. 13), 1170 (FIG. 14), 1190 (FIG. 15), method 1220 (FIG. 16), computing system 1300 (FIG. 17), 1800 (FIG. 18A), computing device 108 (FIG. 18b), pharmacy fulfillment device 1812 (FIG. 19), order processing device 114 (FIG. 20), and method 2100 (FIG. 21). For example, the systems and methods described herein may be configured to identify one or more care pathways (e.g., based on one or more health conditions of the user). In response to identifying the one or more care pathways, the systems and methods described herein may be configured to onboard the user, which may include, based on input received from the user, identifying a provider pool, matching the user with a provider, receiving personal preferences of the user, and determining a mindset of the user.

The systems and methods described herein may be configured to identifying one or more action paths that may include one or more daily interactions (e.g., including one or more queries provided to the user via a personalized experience interface 2200), one or more standard interactions, one or more escalation actions (e.g., based on responses from the user to the daily interactions and/or the standard interactions), one or more emergency actions (e.g., based on responses from the user to the daily interactions and/or the standard interactions), and/or one or more reward actions.

The systems and methods described herein may be configured to provide one or more care visit delivery settings including one or more virtual care visit settings, one or more in home care visit settings, and one or more in-person care visit settings.

The systems and methods described herein may be configured to identify an experience structure that may include curated content, brand content, an experience template, and/or dashboard component construction. The systems and methods described herein may be configured to identifying one or more experience components that may include one or more alerts, content, video content, daily tips, exercises, community components, and/or any other suitable components.

The systems and methods described herein may be configured to generate the personalized experience interface 2200 based on the composite experience components 502. The personalized experience interface 2200 may be configured to provide a personalized experience for engaging the user in actively participating in the treatment, maintenance, therapy, and other aspects of the health of the user.

In some embodiments, the systems and methods described herein may be configured to provide experience structure and experience components. In some embodiments, the systems and methods described herein may be configured to provide identity content based on a user eligibility (e.g., based on a primary care pathway, a secondary care pathway, prescription claim data, electronic medical record data, and/or the like), demographic health persona construct associated with the user (e.g., including general demographics (e.g., age, family history, diagnoses, body mass index, lifestyle factors, social determinants of health, medication, and/or the like) and/or data-driven health dimensions (e.g., general wellbeing, access to technology, stress associated with benefits use, social support, financial comfort and income, trust in healthcare, and/or the like), mindset persona construct of the user (e.g., including persistent denial, angry, fearful, overwhelmed, lonely, independent, inquisitive, determined, and/or the like), self-reported data (e.g., during onboarding) of the user (e.g., including username, electronic mail address, password, social security number, plan or member identification number, one or more models (e.g., standard clinical, relationship focus path, behavioral change focus path, aspirational goals focus path, and/or the like) and/or the like), and/or the like.

In some embodiments, the systems and methods described herein may be configured to receive, responsive to the user filling at least one prescription, a prescription notification indicating information corresponding to the at least one prescription and information corresponding to the user. The information corresponding to the at least one prescription may include at least prescription identification information, dosing information, other suitable information, and the like. The information corresponding to the user may include at least user statistical information, user contact information, other suitable information, and the like.

The systems and methods described herein may be configured to identify, based on the prescription notification, at least one health condition of the user. The systems and methods described herein may be configured to, in response to the at least one health condition corresponding to at least one health condition of a plurality of predetermined health conditions, generate a message including instructions for downloading a user application. The plurality of predetermined health conditions may include at least one of at least one behavioral health condition, at least one musculoskeletal health condition, and/or at least one other health condition. The at least one behavioral health condition may correspond to at least one of depression, generalized anxiety, bipolar disorder, at least one other behavioral health condition, and/or any other suitable behavioral health condition. The at least one musculoskeletal health condition may correspond to at least one of back pain, neck pain, joint pain, at least one other musculoskeletal health condition, and/or any other suitable musculoskeletal health condition. The at least one other health condition corresponds to at least one of cancer, diabetes, heart failure, heart disease, chronic obstructive pulmonary disease, covid 19, at least one other health condition, and/or any other suitable health condition.

The systems and methods described herein may be configured to communicate, to a user account associated with the user, the message. The user account associated with the user may include an electronic mail account, a pharmacy account, a health insurance account, at least one other user account, and/or the like. The systems and methods described herein may be configured to, in response to an indication that the user initiated the user application, provide, at an application setup interface, a plurality of data gathering queries. The data gathering queries may include at least one questions corresponding to a mental or emotional state of the user, a pain level of the user, an energy level of the user, and/or the like. Additionally, or alternatively, the data gathering queries may include one or more questions corresponding to the age of the user, the family history of the user, the location of the user, and/or the like. Additionally, or alternatively, the data gathering queries may correspond to a level of financial comfort of the user, a level of comfort of the user with insurance benefits, demographics of the user, technology access of the user, and/or the like. It should be understood that the data gathering queries may correspond to any suitable information to be gathered from the user.

The systems and methods described herein may be configured to store, in a database or other suitable data store, user responses to the data gathering queries. The systems and methods described herein may be configured to generate, using the user responses, a data structure corresponding to the user. The systems and methods described herein may be configured to generate, based on the data structure corresponding to the user, a personalized experience interface. In some embodiments, the personalized experience interface may comprise a portable health record comprising the health record of the user. The personalized experience interface may include one or more personalized components that include at least a daily interaction component, a relevant resource component, a dynamic selection component, and/or the like. The dynamic selection component may include at least one general selection and/or, responsive to the at least one health condition, at least one health condition specific component.

The systems and methods described herein may be configured to provide, at a display of a computing device associated with the user, the personalized experience interface. The personalized experience interface may correspond to a user avatar that models a biological identity of the user. The user avatar may correspond to personalized, intelligence associated with a predictive healthcare driven model and may be based on a data driven, dynamic patient survey. The systems and methods described herein may be configured to provide, at a metaverse (e.g., a three-dimensional virtual environment), access to at least one three-dimensional environment feature using the user avatar. The at least one three-dimensional environment feature may include a virtual healthcare provider office, a virtual healthcare provider, at least one other three-dimensional environment feature, and/or the like. The virtual healthcare provider may include an avatar corresponding to a healthcare provider (e.g., in the real-world), an avatar corresponding to an artificially intelligent healthcare provider (e.g., a virtual healthcare provider driving my artificial intelligence), and/or the like.

The systems and methods described herein may be configured to identify, using a first set of user responses, a healthcare provider pool. The systems and methods described herein may be configured to adjust at least some of the data gathering queries based on the first set of user responses. The systems and methods described herein may be configured to identify, responsive to a second set of user responses corresponding to at least some of the adjusted data gathering queries, a subset of healthcare providers of the healthcare provider pool that match with the user.

In some embodiments, the personalized experience interface includes, at least, information corresponding to each of the subset of healthcare providers. The systems and methods described herein may be configured to schedule, responsive to a selection of at least one healthcare provider of the subset of healthcare providers by the user using the personalized experience interface, at least one appointment, for the user, with the selected at least one healthcare provider.

The systems and methods described herein may be configured to receive feedback from the user corresponding to at least one of a component of the personalized experience interface and an appointment with the selected at least one healthcare provider. The systems and methods described herein may be configured to refine, responsive to the feedback, the data gathering queries, the personalized experience interface, and/or any other suitable feature. The systems and methods described herein may be configured to identify, based on the feedback, at least one other healthcare provider from the healthcare provider pool.

The systems and methods described herein may be configured to select, using the data structure corresponding to the user, at least one suggestive aspect of the personalized experience interface. The systems and methods described herein may be configured to provide, at the personalized experience interface, the at least one suggestive aspect. The at least one suggestive aspect may include at least one of a color, a dynamic background, at least one other suggestive aspect, and/or the like. Additionally, or alternatively, the at least one suggestive aspect may be configured to provide, responsive to the user engaging with the at least one suggestive aspect, at least one of a physical response of the user and a mental response of the user.

The systems and methods described herein may be configured to generate or refine one or more of at least one aspect of the data gathering queries, at least one aspect of the personalized experience interface, and/or the like, using at least one machine learning model configured to use at least some of the user responses and at least some responses provided by other users to dynamically generate or refine the at least one of at least one aspect of the data gathering queries and at least one aspect of the personalized experience interface.

FIG. 18A is a block diagram of an example implementation of a system 1800 for a high-volume pharmacy. While the system 1800 is generally described as being deployed in a high-volume pharmacy or a fulfillment center (for example, a mail order pharmacy, a direct delivery pharmacy, etc.), the system 1800 and/or components of the system 1800 may otherwise be deployed (for example, in a lower-volume pharmacy, etc.). A high-volume pharmacy may be a pharmacy that is capable of filling at least some prescriptions mechanically. The system 1800 may include a benefit manager device 102 and a pharmacy device 106 in communication with each other directly and/or over a network 104. The system 1800 may also include a storage device 110.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some embodiments, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 1800. In some embodiments, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 1800. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $2200 of annual prescription drug expense, etc.) for certain prescription drugs, certain types and/or classes of prescription drugs, and/or all prescription drugs. The copayment may be stored in the storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 1800) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some embodiments, the amount may also be determined based on other factors. For example, if the member pays the pharmacy for the prescription drug without using the prescription or drug benefit provided by the PBM, the amount of money paid by the member may be higher than when the member uses the prescription or drug benefit. In some embodiments, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 2502.11 standards network, as well as various combinations of the above networks. The network 104 may include an optical network. The network 104 may be a local area network or a global communication network, such as the Internet. In some embodiments, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, or alternatively, in some embodiments, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some embodiments, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 1812, an order processing device 114, and a pharmacy management device 116 in communication with each other directly and/or over the network 104. The order processing device 114 may receive information regarding filling prescriptions and may direct an order component to one or more devices of the pharmacy fulfillment device 1812 at a pharmacy. The pharmacy fulfillment device 1812 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfilment device 1812 to fulfill a prescription and dispense prescription drugs. In some embodiments, the order processing device 114 may be an external order processing device separate from the pharmacy and in communication with other devices located within the pharmacy.

For example, the external order processing device may communicate with an internal pharmacy order processing device and/or other devices located within the system 1800. In some embodiments, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 1812. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some embodiments, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some embodiments, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 1820, claims data 122, drug data 124, prescription data 126, and/or plan sponsor data 128. Further, the system 1800 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some embodiments, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 1820 includes information regarding the members associated with the PBM. The information stored as member data 1820 may include personal information, personal health information, protected health information, etc. Examples of the member data 1820 include name, address, telephone number, e-mail address, prescription drug history, etc. The member data 1820 may include a plan sponsor identifier that identifies the plan sponsor associated with the member and/or a member identifier that identifies the member to the plan sponsor. The member data 1820 may include a member identifier that identifies the plan sponsor associated with the user and/or a user identifier that identifies the user to the plan sponsor. The member data 1820 may also include dispensation preferences such as type of label, type of cap, message preferences, language preferences, etc.

The member data 1820 may be accessed by various devices in the pharmacy (for example, the high-volume fulfillment center, etc.) to obtain information used for fulfillment and shipping of prescription orders. In some embodiments, an external order processing device operated by or on behalf of a member may have access to at least a portion of the member data 1820 for review, verification, or other purposes.

In some embodiments, the member data 1820 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some embodiments, other types of claims beyond prescription drug claims may be stored in the claims data 122. For example, medical claims, dental claims, wellness claims, or other types of health-care-related claims for members may be stored as a portion of the claims data 122.

In some embodiments, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some embodiments, the dosing information reflects a frequency of use (e.g., once a day, twice a day, before each meal, etc.) and a duration of use (e.g., a few days, a week, a few weeks, a month, etc.).

In some embodiments, the order data 118 may be linked to associated member data 1820, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

FIG. 19 illustrates the pharmacy fulfillment device 1812 according to an example implementation. The pharmacy fulfillment device 1812 may be used to process and fulfill prescriptions and prescription orders. After fulfillment, the fulfilled prescriptions are packed for shipping.

The pharmacy fulfillment device 1812 may include devices in communication with the benefit manager device 102, the order processing device 114, and/or the storage device 110, directly or over the network 104. Specifically, the pharmacy fulfillment device 1812 may include pallet sizing and pucking device(s) 206, loading device(s) 208, inspect device(s) 2210, unit of use device(s) 212, automated dispensing device(s) 214, manual fulfillment device(s) 216, review devices 218, imaging device(s) 2220, cap device(s) 222, accumulation devices 224, packing device(s) 226, literature device(s) 228, unit of use packing device(s) 2230, and mail manifest device(s) 232. Further, the pharmacy fulfillment device 1812 may include additional devices, which may communicate with each other directly or over the network 104.

In some embodiments, operations performed by one of these devices 206-232 may be performed sequentially, or in parallel with the operations of another device as may be coordinated by the order processing device 114. In some embodiments, the order processing device 114 tracks a prescription with the pharmacy based on operations performed by one or more of the devices 206-232.

In some embodiments, the pharmacy fulfillment device 1812 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In some embodiments, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 2210 may verify that containers in a pallet are correctly labeled and in the correct spot on the pallet. The inspect device 2210 may scan the label on one or more containers on the pallet. Labels of containers may be scanned or imaged in full or in part by the inspect device 2210. Such imaging may occur after the container has been lifted out of its puck by a robotic arm, picker, etc., or may be otherwise scanned or imaged while retained in the puck. In some embodiments, images and/or video captured by the inspect device 2210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some embodiments, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some embodiments, the manual fulfillment device 216 provides the filled container to another device in the pharmacy fulfillment devices 1812 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 2220 may image containers once they have been filled with pharmaceuticals. The imaging device 2220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some embodiments, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some embodiments, the literature device 228 folds or otherwise prepares the literature for inclusion with a prescription drug order (e.g., in a shipping container). In some embodiments, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 2230 packages a unit of use prescription order in preparation for shipping the order. The unit of use packing device 2230 may include manual scanning of containers to be bagged for shipping to verify each container in the order. In some embodiments, the manual scanning may be performed at a manual scanning station. The pharmacy fulfillment device 1812 may also include a mail manifest device 232 to print mailing labels used by the packing device 226 and may print shipping manifests and packing lists.

While the pharmacy fulfillment device 1812 in FIG. 19 is shown to include single devices 206-232, multiple devices may be used. When multiple devices are present, the multiple devices may be of the same device type or models, or may be a different device type or model. The types of devices 206-232 shown in FIG. 19 are example devices. In other configurations of the system 1800, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

FIG. 20 illustrates the order processing device 114 according to some embodiments. The order processing device 114 may be used by one or more operators to generate prescription orders, make routing decisions, make prescription order consolidation decisions, track literature with the system 1800, and/or view order status and other order related information. For example, the prescription order may include order components.

The order processing device 114 may receive instructions to fulfill an order without operator intervention. An order component may include a prescription drug fulfilled by use of a container through the system 1800. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 1800. In some embodiments, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, and the like. The order tracking subsystem 306 may store data locally (for example, in a memory) or as a portion of the order data 118 stored in the storage device 110.

In some embodiments, the system 1800 may include one or more computing devices 108, as is generally illustrated in FIG. 18B. The computing device 108 may include any suitable computing device, such as a mobile computing device, a desktop computing device, a laptop computing device, a server computing device, other suitable computing device, or a combination thereof. The computing device 108 may be used by a user accessing the pharmacy associated with the system 1800, as described. Additionally, or alternatively, the computing device 108 may be configured to identify an optimum or substantially optimum combination of data objects, as described.

The computing device 108 may include a processor 1830 configured to control the overall operation of computing device 108. The processor 1830 may include any suitable processor, such as those described herein. The computing device 108 may also include a user input device 132 that is configured to receive input from a user of the computing device 108 and to communicate signals representing the input received from the user to the processor 1830. For example, the user input device 132 may include a button, keypad, dial, touch screen, audio input interface, visual/image capture input interface, input in the form of sensor data, etc.

The computing device 108 may include a display 136 that may be controlled by the processor 1830 to display information to the user. A data bus 138 may be configured to facilitate data transfer between, at least, a storage device 140 and the processor 1830. The computing device 108 may also include a network interface 142 configured to couple or connect the computing device 108 to various other computing devices or network devices via a network connection, such as a wired or wireless connection, such as the network 104. In some embodiments, the network interface 142 includes a wireless transceiver.

The storage device 140 may include a single disk or a plurality of disks (e.g., hard drives), one or more solid-state drives, one or more hybrid hard drives, and the like. The storage device 140 may include a storage management module that manages one or more partitions within the storage device 140. In some e embodiments, storage device 140 may include flash memory, semiconductor (solid state) memory or the like. The computing device 108 may also include a memory 144. The memory 144 may include Random Access Memory (RAM), a Read-Only Memory (ROM), or a combination thereof. The memory 144 may store programs, utilities, or processes to be executed in by the processor 1830. The memory 144 may provide volatile data storage, and stores instructions related to the operation of the computing device 108. In some embodiments, the processor 1830 may be configured to execute instructions stored on the memory 144 to, at least, perform various aspects of the systems and methods described herein.

In some embodiments, the computing device 108 may include an artificial intelligence engine 146 configured to use one or more machine learning models 148 configured to perform at least some aspects of the systems and methods described herein. The artificial intelligence engine 146 may include any suitable artificial intelligence engine and may be disposed on computing device 108 or remotely located from the computing device 108, such as in a cloud computing device or other suitable remotely located computing device. The computing device 108 may include a training engine capable of generating the one or more machine learning models 148. The one or more machine learning models 148 may be trained using any suitable data, including those described herein. Additionally, or alternatively, the one or more machine learning models 148 may be iteratively trained (e.g., subsequent to an initiate training) using output from the one or more machine learning models 148 (e.g., as feedback, which may or may not include input from a user indicating accuracy of one or more predictions associated with the output of the one or more machine learning models 148).

In some embodiments, the computing device 108 may be configured to receive, responsive to the user filling at least one prescription, a prescription notification indicating information corresponding to the at least one prescription and information corresponding to the user. The information corresponding to the at least one prescription may include at least prescription identification information, dosing information, other suitable information, and the like. The information corresponding to the user may include at least user statistical information, user contact information, other suitable information, and the like.

The computing device 108 may identify, based on the prescription notification, at least one health condition of the user. The computing device 108 may, in response to the at least one health condition corresponding to at least one health condition of a plurality of predetermined health conditions, generate a message including instructions for downloading a user application (e.g., at a computing device associated with the user). The plurality of predetermined health conditions may include at least one of at least one behavioral health condition, at least one musculoskeletal health condition, and/or at least one other health condition. The at least one behavioral health condition may correspond to at least one of depression, generalized anxiety, bipolar disorder, at least one other behavioral health condition, and/or any other suitable behavioral health condition. The at least one musculoskeletal health condition may correspond to at least one of back pain, neck pain, joint pain, at least one other musculoskeletal health condition, and/or any other suitable musculoskeletal health condition. The at least one other health condition corresponds to at least one of cancer, diabetes, heart failure, heart disease, chronic obstructive pulmonary disease, covid 19, at least one other health condition, and/or any other suitable health condition.

The computing device 108 may communicate (e.g., via the network interface 142 or other suitable interface), to a user account associated with the user, the message. The user account associated with the user may include an electronic mail account, a pharmacy account, a health insurance account, at least one other user account, and/or the like. The computing device 108 may, in response to an indication that the user initiated the user application, provide, at an application setup interface associated with the user application (e.g., provided at the computing device associated with the user), a plurality of data gathering queries. The data gathering queries may include at least one questions corresponding to a mental or emotional state of the user, a pain level of the user, an energy level of the user, and/or the like. Additionally, or alternatively, the data gathering queries may include one or more questions corresponding to the age of the user, the family history of the user, the location of the user, and/or the like. Additionally, or alternatively, the data gathering queries may correspond to a level of financial comfort of the user, a level of comfort of the user with insurance benefits, demographics of the user, technology access of the user, and/or the like. It should be understood that the data gathering queries may correspond to any suitable information to be gathered from the user.

The computing device 108 may store, in a database or other suitable data store (e.g., such as the storage device 110, the storage device 140, and/or any other suitable device), user responses to the data gathering queries. The computing device 108 may generate, using the user responses, a data structure corresponding to the user. The computing device 108 may generate, based on the data structure corresponding to the user, a personalized experience interface, such as the personalize experience interface 2200. In some embodiments, the personalized experience interface 2200 may comprise a portable health record comprising the health record of the user. The personalized experience interface 2200 may include one or more personalized components that include at least a daily interaction component, a relevant resource component, a dynamic selection component, and/or the like. The dynamic selection component may include at least one general selection and/or, responsive to the at least one health condition, at least one health condition specific component.

The computing device 108 may provide, at a display of the computing device associated with the user or other suitable display, the personalized experience interface 2200. The personalized experience interface 2200 may correspond to a user avatar that models a biological identity of the user. The user avatar may correspond to personalized, intelligence associated with a predictive healthcare driven model and may be based on a data driven, dynamic patient survey. The computing device 108 may provide, at a metaverse (e.g., a three-dimensional virtual environment), access to at least one three-dimensional environment feature using the user avatar. The at least one three-dimensional environment feature may include a virtual healthcare provider office, a virtual healthcare provider, at least one other three-dimensional environment feature, and/or the like. The virtual healthcare provider may include an avatar corresponding to a healthcare provider (e.g., in the real-world), an avatar corresponding to an artificially intelligent healthcare provider (e.g., a virtual healthcare provider driving my artificial intelligence), and/or the like.

The computing device 108 may identify, using a first set of user responses, a healthcare provider pool. The computing device 108 may adjust at least some of the data gathering queries based on the first set of user responses. The computing device 108 may identify, responsive to a second set of user responses corresponding to at least some of the adjusted data gathering queries, a subset of healthcare providers of the healthcare provider pool that match with the user.

In some embodiments, the personalized experience interface 2200 may include, at least, information corresponding to each of the subset of healthcare providers. The computing device 108 may schedule, responsive to a selection of at least one healthcare provider of the subset of healthcare providers by the user using the personalized experience interface 2200, at least one appointment, for the user, with the selected at least one healthcare provider.

The computing device 108 may receive feedback from the user corresponding to at least one of a component of the personalized experience interface 2200, an appointment with the selected at least one healthcare provider and/or any other suitable aspect of the systems and methods described herein. The computing device 108 may refine, responsive to the feedback, the data gathering queries, the personalized experience interface 2200, and/or any other suitable feature. The computing device 108 may identify, based on the feedback, at least one other healthcare provider from the healthcare provider pool.

The computing device 108 may select, using the data structure corresponding to the user, at least one suggestive aspect of the personalized experience interface 2200. The computing device 108 may provide, at the personalized experience interface 2200, the at least one suggestive aspect. The at least one suggestive aspect may include at least one of a color, a dynamic background, at least one other suggestive aspect, and/or the like. Additionally, or alternatively, the at least one suggestive aspect may be configured to provide, responsive to the user engaging with the at least one suggestive aspect, at least one of a physical response of the user and a mental response of the user.

The computing device 108 may generate or refine one or more of at least one aspect of the data gathering queries, at least one aspect of the personalized experience interface 2200, and/or the like, using at least one machine learning model, such as the machine learning model 148 or other suitable machine learning model. The machine learning model 148 may be configured to use at least some of the user responses and at least some responses provided by other users to dynamically generate or refine the at least one of at least one aspect of the data gathering queries, at least one aspect of the personalized experience interface 2200, and/or any other suitable feature or aspect of the systems and methods described herein.

In some embodiments, the computing device 108 and/or the system 1800 may perform the methods described herein. However, the methods described herein as performed by the computing device 108 and/or the system 1800 are not meant to be limiting, and any type of software executed on a computing device or a combination of various computing devices can perform the methods described herein without departing from the scope of this disclosure.

FIG. 21 is a flow diagram generally illustrating personalized healthcare experience method 2100 according to the principles of the present disclosure. At 402, the method 2100 receives, responsive to a user filling at least one prescription, a prescription notification indicating information corresponding to the at least one prescription and information corresponding to the user. For example, the computing device 108 may receive, responsive to the user filling the at least one prescription, the prescription notification.

At 404, the method 2100 identifies, based on the prescription notification, at least one health condition of the user. For example, the computing device 108 may identify, based on the prescription notification, the at least one health condition of the user. The at least one health condition of the user may include a behavioral health condition or other suitable condition.

At 406, the method 2100, in response to the at least one health condition corresponding to at least one health condition of a plurality of predetermined health conditions, generates a message including instructions for downloading a user application. For example, the computing device 108 may generate, in response to the at least one health condition corresponding to at least one health condition of a plurality of predetermined health conditions, the message including instructions for downloading the user application.

At 408, the method 2100 communicates, to a user account associated with the user, the message. For example, the computing device 108 may communicate the message to the user account associated with the user.

At 2110, the method 2100, in response to an indication that the user initiated the user application, provides, at an application setup interface, a plurality of data gathering queries. For example, the computing device 108 may, in response to the indication that the user initiated the user application, provide, at the application setup interface, the plurality of data queries.

At 412, the method 2100 stores user responses to the data gathering queries. For example, the computing device 108 may store the user responses to the data gathering queries.

At 414, the method 2100 generates, using the user responses, a data structure corresponding to the user. For example, the computing device 108 may generate, using the user responses, the data structure corresponding to the user.

At 416, the method 2100 generates, based on the data structure corresponding to the user, a personalized experience interface. For example, the computing device 108 may generate, based on the data structure corresponding to the user, the personalized experience interface 2200. The personalized experience interface 2200 may be configured to provide behavioral health components, such as sleep trackers, exercise trackers, daily mental health tips, and the like. Additionally, or alternatively, the personalized experience interface 2200 may provide daily queries to the user (e.g., requesting the user provide input indicating how the user is feeling, how the user has been felt over a period of time, what the user's mental state is, and/or various other information associated with the user). The computing device 108 may use input provided by the user to take one or more actions. For example, if the user provides input (e.g., based on a signal response and/or a combination of multiple responses) that indicates a certain behavioral concern (e.g., self-harm or other suitable behavioral concern), the computing device 108 may generate an alert to a human or artificial intelligence agent to contact the user and/or a healthcare provider.

At 418, the method 2100 provides, at a display of a computing device associated with the user, the personalized experience interface. For example, the computing device 108 may provide, at the display of the computing device associated with the user, the personal experience interface 2200.

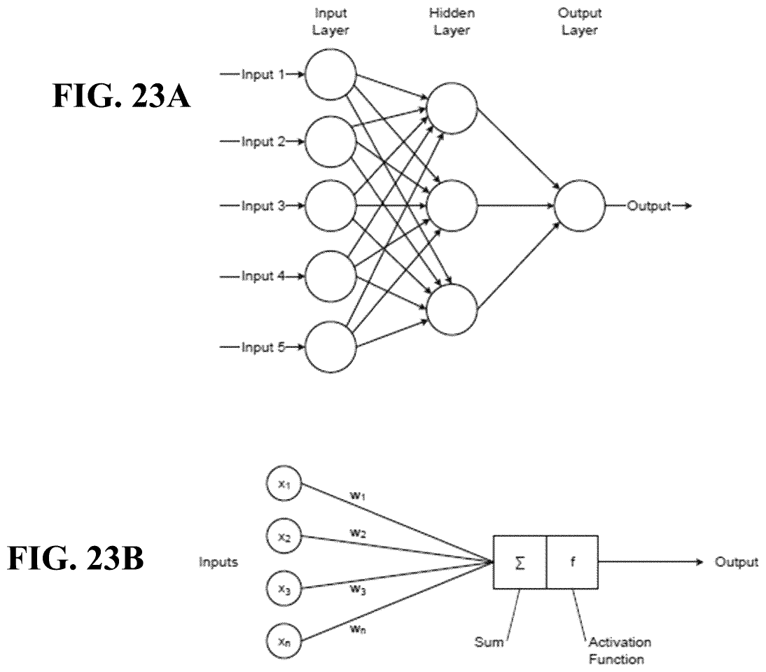

FIG. 23A shows a fully connected neural network (e.g., which may be associated with the machine learning model 148), where each neuron in a given layer is connected to each neuron in a next layer. In the input layer, each input node is associated with a numerical value, which can be any real number. The neural network may be used to perform at least some of the features of the systems and methods described herein. In each layer, each connection that departs from an input node has a weight associated with it, which can also be any real number (see FIG. 23B). In the input layer, the number of neurons equals number of features (columns) in a dataset. The output layer may have multiple continuous outputs.

The layers between the input and output layers are hidden layers. The number of hidden layers can be one or more (one hidden layer may be sufficient for most applications). A neural network with no hidden layers can represent linear separable functions or decisions. A neural network with one hidden layer can perform continuous mapping from one finite space to another. A neural network with two hidden layers can approximate any smooth mapping to any accuracy.

The number of neurons can be optimized. At the beginning of training, a network configuration is more likely to have excess nodes. Some of the nodes may be removed from the network during training that would not noticeably affect network performance. For example, nodes with weights approaching zero after training can be removed (this process is called pruning). The number of neurons can cause under-fitting (inability to adequately capture signals in dataset) or over-fitting (insufficient information to train all neurons; network performs well on training dataset but not on test dataset).

Various methods and criteria can be used to measure performance of a neural network model. For example, root mean squared error (RMSE) measures the average distance between observed values and model predictions. Coefficient of Determination (R2) measures correlation (not accuracy) between observed and predicted outcomes. This method may not be reliable if the data has a large variance. Other performance measures include irreducible noise, model bias, and model variance. A high model bias for a model indicates that the model is not able to capture true relationship between predictors and the outcome. Model variance may indicate whether a model is stable (a slight perturbation in the data will significantly change the model fit). The neural network can receive inputs, e.g., vectors, that can be used to generate models that can be used with provider matching, risk model processing, or both, as described herein.

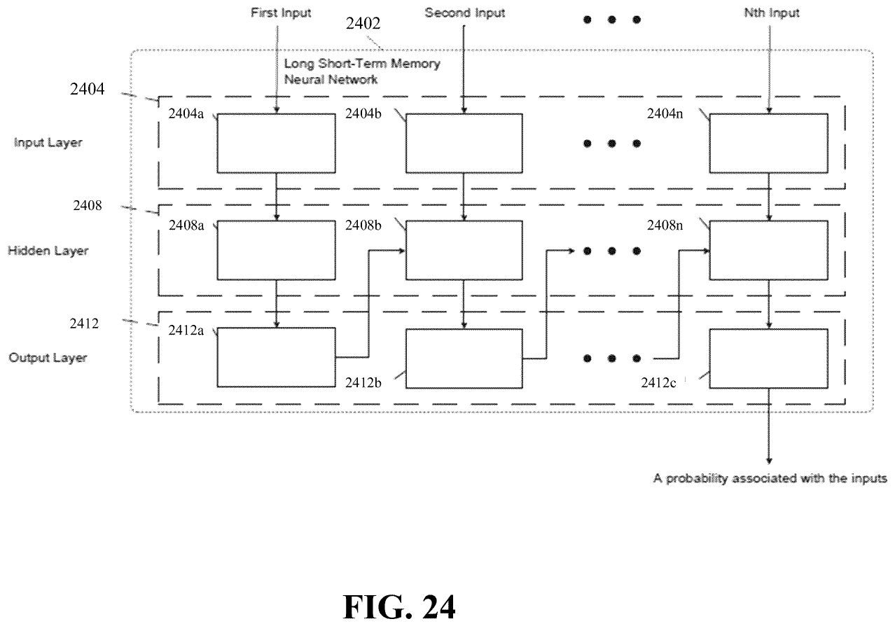

FIG. 24 illustrates an example of a long short-term memory (LSTM) neural network 2402 used to generate models such as those described above, using machine learning techniques. The LSTM neural network 2402 may be used to implement a machine learning model, such as the machine learning model 148 or other suitable machine learning model, and, in some embodiments, may use other types of machine learning networks. The LSTM network 2402 may be used to implement at least some of the features of the systems and methods described herein. The LSTM neural network 2402 includes an input layer 2404, a hidden layer 2408, and an output layer 2412. The input layer 2404 includes inputs 2404a, 2404b . . . 2404n. The hidden layer 2408 includes neurons 2408a, 2408b . . . 2408n. The output layer 2412 includes outputs 2412a, 2412b . . . 2412n.

Each neuron of the hidden layer 2408 receives an input, such as those described with respect to FIGS. 23A-23B from the input layer 2404 and outputs a value to the corresponding output in the output layer 2412 (e.g., including, but not limited to, one or more outputs corresponding to the generation or refinement of one or more of at least one aspect of the data gathering queries, at least one aspect of the personalized experience interface 2200, and/or the like, as described). For example, the neuron 2408a receives an input from the input 2404a and outputs a value to the output 2412a. Each neuron, other than the neuron 2408a, also receives an output of a previous neuron as an input. For example, the neuron 2408b receives inputs from the input 2404b and the output 2412a. In this way the output of each neuron is fed forward to the next neuron in the hidden layer 2408. The last output 2412n in the output layer 2412 outputs a probability associated with the inputs 2404a-2404n. Although the input layer 2404, the hidden layer 2408, and the output layer 2412 are depicted as each including three elements, each layer may contain any number of elements.

In some embodiments, each layer of the LSTM neural network 2402 must include the same number of elements as each of the other layers of the LSTM neural network 2402. In some embodiments, a convolutional neural network may be implemented. Similar to LSTM neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 404a is connected to each of neurons 2408a, 2408b . . . 2408n.

In some embodiments, each input node in the input layer may be associated with a numerical value, which can be any real number. In each layer, each connection that departs from an input node has a weight associated with it, which can also be any real number. In the input layer, the number of neurons equals number of features (columns) in a dataset. The output layer may have multiple continuous outputs.

As mentioned above, the layers between the input and output layers are hidden layers. The number of hidden layers can be one or more (one hidden layer may be sufficient for many applications). A neural network with no hidden layers can represent linear separable functions or decisions. A neural network with one hidden layer can perform continuous mapping from one finite space to another. A neural network with two hidden layers can approximate any smooth mapping to any accuracy. The neural network of FIG. 24 can receive inputs, e.g., vectors, that can be used to generate models that can be used with provider matching, risk model processing, or both, as described herein.

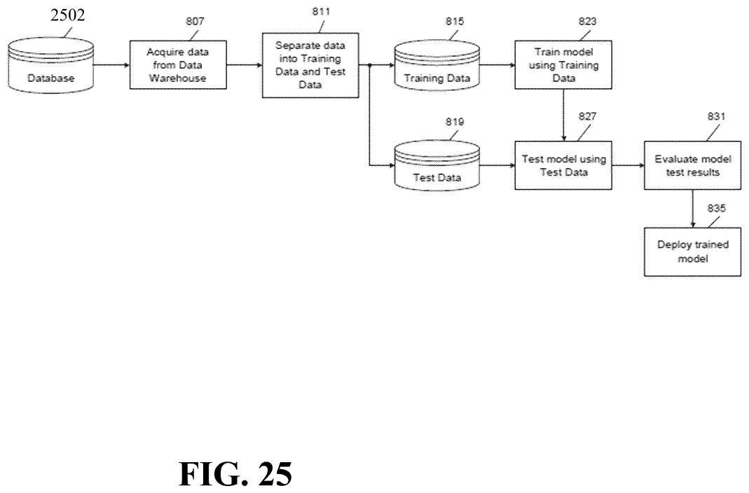

FIG. 25 illustrates an example process for generating a machine learning model, including, but not limited to, the machine learning model 148. At 807, control obtains data from a database 2502 (e.g., a data warehouse), such as the database 402. The data may include any suitable data for developing machine learning models, such as the machine learning model 148 and/or any other suitable machine learning models.

At 811, control separates the data obtained from the database 2502 into training data 815 and test data 819. The training data 815 is used to train the model at 823, and the test data 819 is used to test the model at 827. Typically, the set of training data 815 is selected to be larger than the set of test data 819, depending on the desired model development parameters. For example, the training data 815 may include about seventy percent of the data acquired from the database 2502, about eighty percent of the data, about ninety percent, etc. The remaining thirty percent, twenty percent, or ten percent, is then used as the test data 819.

Separating a portion of the acquired data as test data 819 allows for testing of the trained model against actual output data, to facilitate more accurate training and development of the model at 823 and 827. The model may be trained at 823 using any suitable machine learning model techniques, including those described herein, such as random forest, generalized linear models, decision tree, and neural networks.

At 831, control evaluates the model test results. For example, the trained model may be tested at 827 using the test data 819, and the results of the output data from the tested model may be compared to actual outputs of the test data 819, to determine a level of accuracy. The model results may be evaluated using any suitable machine learning model analysis, such as the example techniques described further below.

After evaluating the model test results at 831, the model may be deployed at 835 if the model test results are satisfactory. Deploying the model may include using the model to make predictions for a large-scale input dataset with unknown outputs. If the evaluation of the model test results at 831 is unsatisfactory, the model may be developed further using different parameters, using different modeling techniques, using other model types, etc. The machine learning model method of FIG. 25 can receive inputs, e.g., vectors, that can be used to generate models that can be used with provider matching, risk model processing, or both, as described herein.

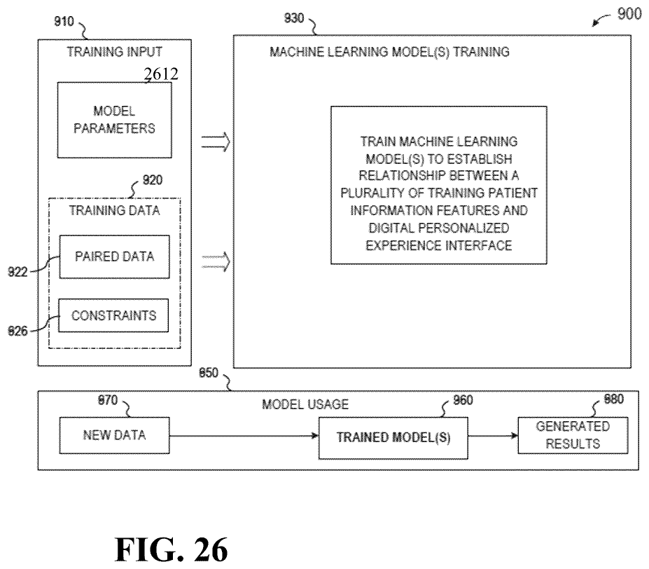

FIG. 26 is a block diagram of an example an interactive digital personalized experience interface system 900 that may be deployed within the system of FIG. 1, according to some embodiments. Training input 910 includes model parameters 912 and training data 920 (e.g., training data related to user interfaces, behavioral health data, and/or the like) which may include paired training data sets 922 (e.g., input-output training pairs) and constraints 926. Model parameters 912 stores or provides the parameters or coefficients of corresponding ones of machine learning models. During training, these parameters 912 are adapted based on the input-output training pairs of the training data sets 922. After the parameters 912 are adapted (after training), the parameters are used by trained models 960 to implement the trained machine learning models on a new set of data 970.

Training data 920 includes constraints 926 which may define the constraints of a given patient information features. The paired training data sets 922 may include sets of input-output pairs, such as a pairs of a plurality of training patient information features and types of service of care associated with the training patient information features. Some components of training input 910 may be stored separately at a different off-site facility or facilities than other components.

Machine learning model(s) training 930 trains one or more machine learning techniques based on the sets of input-output pairs of paired training data sets 922. For example, the model training 930 may train the machine learning model parameters 912 by minimizing a loss function based on one or more ground-truth type of service of care. The machine learning model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, the machine learning model can be applied to a training plurality of patient information features to estimate or generate a prediction of a type of service of care. In some embodiments, a derivative of a loss function is computed based on a comparison of the estimated type of service of care and the ground truth type of service of care associated with the training patient information features and parameters of the machine learning model are updated based on the computed derivative of the loss function.

In some embodiments, the machine learning model receives a batch of training data that includes a first set of the plurality of training patient information features together with a ground-truth type of service of care associated with the first set of the plurality of training patient information features. The machine learning model generates a feature vector based on the first set of the plurality of training patient information features and generates a prediction of one or more types of service of care. The prediction is compared with the ground truth type of service of care and parameters of the machine learning model are updated based on the comparison.

The result of minimizing the loss function for multiple sets of training data trains, adapts, or optimizes the model parameters 912 of the corresponding machine learning models. In this way, the machine learning model is trained to establish a relationship between a plurality of training patient information features and types of service of care.

After the machine learning model is trained, new data 970, including one or more patient information features are received. The trained machine learning technique may be applied to the new data 970 to generate results 980 including a prediction of a service of care type to recommend. The selection or recommendation made by the system 156 can be represented in a graphical user interface that depicts each of a plurality of different types of service of care or other patient interactive interface.

In some embodiments, a method for providing an interactive digital personalized experience interface includes receiving, responsive to a user filling at least one prescription, a prescription notification indicating information corresponding to the at least one prescription and information corresponding to the user. The method also includes identifying, based on the prescription notification, at least one health condition of the user. The method also includes, in response to the at least one health condition corresponding to at least one health condition of a plurality of predetermined health conditions, generating a message including instructions for downloading a user application. The method also includes communicating, to a user account associated with the user, the message and, in response to an indication that the user initiated the user application, providing, at an application setup interface, a plurality of data gathering queries. The method also includes storing user responses to the data gathering queries and generating, using the user responses, a data structure corresponding to the user. The method also includes generating, based on the data structure corresponding to the user, a personalized experience interface, and providing, at a display of a computing device associated with the user, the personalized experience interface.

In some embodiments, the information corresponding to the at least one prescription includes at least prescription identification information and dosing information. In some embodiments, the information corresponding to the user includes at least user statistical information and user contact information. In some embodiments, the plurality of predetermined health conditions include at least one of at least one behavioral health condition, at least one musculoskeletal health condition, and at least one other health condition. In some embodiments, the at least one behavioral health condition corresponds to at least one of depression, generalized anxiety, bipolar disorder, and at least one other behavioral health condition. In some embodiments, the at least one musculoskeletal health condition corresponds to at least one of back pain, neck pain, joint pain, and at least one other musculoskeletal health condition. In some embodiments, the at least one other health condition corresponds to at least one of cancer, diabetes, heart failure, heart disease, chronic obstructive pulmonary disease, covid 19, and at least one other health condition.

In some embodiments, the user account associated with the user includes at least one of an electronic mail account, a pharmacy account, a health insurance account, and at least one other user account. In some embodiments, the data gathering queries include at least one questions corresponding to, at least, a mental or emotional state of the user. In some embodiments, the personalized experience interface includes one or more personalized components that include at least a daily interaction component, a relevant resource component, and a dynamic selection component. In some embodiments, the dynamic selection component includes at least one general selection and, responsive to the at least one health condition, at least one health condition specific component. In some embodiments, the personalized experience interface corresponds to a user avatar that models a biological identity of the user.

In some embodiments, the method also includes providing, at a metaverse, access to at least one three-dimensional environment feature using the user avatar. In some embodiments, the at least one three-dimensional environment feature includes at least one of a virtual healthcare provider office, a virtual healthcare provider, and at least one other three-dimensional environment feature. In some embodiments, the virtual healthcare provider includes at least one of an avatar corresponding to a healthcare provider and an avatar corresponding to an artificially intelligent healthcare provider. In some embodiments, the method also includes identifying, using a first set of user responses, a healthcare provider pool. In some embodiments, the method also includes adjusting at least some of the data gathering queries based on the first set of user responses. In some embodiments, the method also includes identifying, responsive to a second set of user responses corresponding to at least some of the adjusted data gathering queries, a subset of healthcare providers of the healthcare provider pool that match with the user.

In some embodiments, the personalized experience interface includes, at least, information corresponding to each of the subset of healthcare providers. In some embodiments, the method also includes scheduling, responsive to a selection of at least one healthcare provider of the subset of healthcare providers by the user using the personalized experience interface, at least one appointment, for the user, with the selected at least one healthcare provider. In some embodiments, the method also includes receiving feedback from the user corresponding to at least one of a component of the personalized experience interface and an appointment with the selected at least one healthcare provider. In some embodiments, the method also includes refining, responsive to the feedback, at least the data gathering queries and the personalized experience interface. In some embodiments, the method also includes identifying, based on the feedback, at least one other healthcare provider from the healthcare provider pool.

In some embodiments, the method also includes selecting, using the data structure corresponding to the user, at least one suggestive aspect of the personalized experience interface and providing, at the personalized experience interface, the at least one suggestive aspect. In some embodiments, the at least one suggestive aspect includes at least one of a color, a dynamic background, and at least one other suggestive aspect. In some embodiments, the at least one suggestive aspect is configured to provide, responsive to the user engaging with the at least one suggestive aspect, at least one of a physical response of the user and a mental response of the user. In some embodiments, the method also includes generating or refining at least one of at least one aspect of the data gathering queries and at least one aspect of the personalized experience interface, using at least one machine learning model configured to use at least some of the user responses and at least some responses provided by other users to dynamically generate or refine the at least one of at least one aspect of the data gathering queries and at least one aspect of the personalized experience interface.

In some embodiments, a system for providing an interactive digital personalized experience interface includes a processor, and a memory. The memory includes instructions that, when executed by the processor, cause the processor to: receive, responsive to a user filling at least one prescription, a prescription notification indicating information corresponding to the at least one prescription and information corresponding to the user; identify, based on the prescription notification, at least one health condition of the user; in response to the at least one health condition corresponding to at least one health condition of a plurality of predetermined health conditions, generate a message including instructions for downloading a user application; communicate, to a user account associated with the user, the message; in response to an indication that the user initiated the user application, provide, at an application setup interface, a plurality of data gathering queries; store user responses to the data gathering queries; generate, using the user responses, a data structure corresponding to the user; generate, based on the data structure corresponding to the user, a personalized experience interface; and provide, at a display of a computing device associated with the user, the personalized experience interface.

In some embodiments, the instructions further cause the processor to identify, using a first set of user responses, a healthcare provider pool. In some embodiments, the instructions further cause the processor to adjust at least some of the data gathering queries based on the first set of user responses. In some embodiments, the instructions further cause the processor to identify, responsive to a second set of user responses corresponding to at least some of the adjusted data gathering queries, a subset of healthcare providers of the healthcare provider pool that match with the user.

In some embodiments, an apparatus for providing an interactive digital personalized experience interface includes one or more processors, and a memory. The memory includes instructions that, when executed by the one or more processors, cause the one or more processors to, respectively or collectively: receive a prescription notification indicating information corresponding to at least one prescription and information corresponding to a user associated with the at least one prescription; identify, based on the prescription notification, at least one health condition of the user; in response to the at least one health condition corresponding to at least one health condition of a plurality of predetermined health conditions, generate a message including instructions for downloading a user application; communicate, to a user account associated with the user, the message; in response to an indication that the user initiated the user application, provide, at an application setup interface, a plurality of data gathering queries; generate, using the user responses, a data structure corresponding to the user; generate, based on the data structure corresponding to the user, a personalized experience interface; and provide, at a display of a computing device associated with the user, the personalized experience interface.

It will be understood that while certain examples and embodiments are specifically described as being combinable, it is understood that any of the examples and embodiments described herein are combinable with each other whether explicitly stated or not.

The present disclosure relates generally to interactive digital personalized experience interfaces which can be used with various other digital healthcare systems and methods such as U.S. patents application Ser. No. 18/204,616, titled METHODS AND SYSTEMS FOR UPDATING AND CURATING DATA; Ser. No. 18/204,716, titled RECURRING REMOTE MONITORING WITH REAL-TIME EXCHANGE TO ANALYZE HEALTH DATA AND GENERATE ACTION PLANS; Ser. No. 18/204,798, titled SURVEY AND SUGGESTION SYSTEM, Ser. No. 18/204,834, titled AUTOMATED RISK MODEL PROCESSING AND MULTIDIMENSIONAL PROVIDER MATCHING ARCHITECTURE; Ser. No. 18/205,203, titled MACHINE LEARNING MODELS FOR GENERATING EXECUTABLE SEQUENCES; and Ser. No. 18/205,249, titled SCALABLE FRAMEWORK FOR DIGITAL MESH, each of which are incorporated by reference. The predictive model generation systems can be used with the present disclosure to generate or interpret the interactive digital personalized experience interfaces.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 2502.11-2016 (also known as the WIFI wireless networking standard) and IEEE Standard 2502.3-2015 (also known as the ETHERNET wired networking standard). Examples of a WPAN are the BLUETOOTH wireless networking standard from the Bluetooth Special Interest Group and IEEE Standard 2502.15.4.

The module may communicate with other modules using the interface circuit(s). Although the module may be depicted in the present disclosure as logically communicating directly with other modules, in some embodiments the module may actually communicate via a communications system. The communications system includes physical and/or virtual networking equipment such as hubs, switches, routers, and gateways. In some embodiments, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In some embodiments, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Implementations of the systems, algorithms, methods, instructions, etc., described herein may be realized in hardware, software, or any combination thereof. The hardware may include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

### Glossary

“CARRIER SIGNAL” in this context refers to any intangible medium that is capable of storing, encoding, or carrying transitory or non-transitory instructions for execution by the machine, and includes digital or analog communications signals or other intangible medium to facilitate communication of such instructions. Instructions may be transmitted or received over the network using a transitory or non-transitory transmission medium via a network interface device and using any one of a number of well-known transfer protocols.

“CLIENT DEVICE” in this context refers to any machine that interfaces to a communications network to obtain resources from one or more server systems or other client devices. A client device may be, but is not limited to, a mobile phone, desktop computer, laptop, PDA, smart phone, tablet, ultra-book, netbook, laptop, multi-processor system, microprocessor-based or programmable consumer electronics, game console, set-top box, or any other communication device that a user may use to access a network.

“COMMUNICATIONS NETWORK” in this context refers to one or more portions of a network that may be an ad hoc network, an intranet, an extranet, a virtual private network (VPN), a LAN, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an ASIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC, or any combination thereof. A processor may further be a multi-core processor having two or more independent processors (sometimes referred to as “cores”) that may execute instructions contemporaneously.

“TIMESTAMP” in this context refers to a sequence of characters or encoded information identifying when a certain event occurred, for example giving date and time of day, sometimes accurate to a small fraction of a second.

Changes and modifications may be made to the disclosed embodiments without departing from the scope of the present disclosure. These and other changes or modifications are intended to be included within the scope of the present disclosure, as expressed in the following claims.

The Abstract of the Disclosure is provided to comply with 37 C.F.R. § 1.72 (b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. At least one computer readable, non-transitory storage medium comprising a set of executable program instructions, which when executed by a computing system, cause the computing system to:
select a given group of monitoring information to collect from a user, wherein the given group of monitoring information includes prescription information;
instruct a first computing device associated with the user to collect the given group of monitoring information;
determine that the given group of monitoring information is unavailable to be provided by the first computing device with the given group of monitoring information being used to identify at least one health condition of the user;
determine a class associated with the first computing device;
apply a machine learning model to the class associated with the first computing device to determine whether to instruct the first computing device to re-collect the given group of monitoring information;
instruct the first computing device to re-collect the given group of monitoring information based on the class associated with the first computing device and the machine learning model determining that the first computing device is to re-collect the given group of monitoring information;
obtain a batch of training data comprising a first set of a plurality of training computing device class features associated with re-reading requests;
process the first set of the plurality of training computing device class features by the machine learning model to generate an estimated need for a re-reading request;
compute a loss based on a deviation between the estimated need for the re-reading request and the re-reading requests associated with the first set of the plurality of training computing device class features;
update parameters of the machine learning model based on the computed loss;
in response to the given group of monitoring information being determined as being unavailable to be provided by the first computing device, generate an outreach event to request the given group of monitoring information from the user; and
in response to the given group of monitoring information being determined as being available, select an action pathway for the user based at least in part on the prescription information.

2. The at least one computer readable storage medium of claim 1, wherein the instructions, when executed, further cause the computing system to:
receive, responsive to a user fulfilling at least one prescription, a prescription notification indicating information corresponding to the at least one prescription and information corresponding to the user; and
identify, based on the prescription notification, at least one health condition of the user.

3. The at least one computer readable storage medium of claim 2, wherein the instructions, when executed, further cause the computing system to:
in response to the at least one health condition corresponding to at least one health condition of a plurality of predetermined health conditions, generate a message including instructions for downloading a user application; and
communicate, to a user account associated with the user, the message.

4. The at least one computer readable storage medium of claim 3, wherein the instructions, when executed, further cause the computing system to:
in response to an indication that the user initiated the user application, provide, at an application setup interface, a plurality of data gathering queries;
store user responses to the data gathering queries;
generate, using the user responses, a data structure corresponding to the user; and
generate, based on the data structure corresponding to the user, a personalized experience interface that includes a user avatar that models, based on at least the information corresponding to the user and the at least one prescription, a biological identity of the user.

5. The at least one computer readable storage medium of claim 4, wherein the instructions, when executed, further cause the computing system to:
provide, at a display of the first computing device associated with the user, the personalized experience interface.

6. The at least one computer readable storage medium of claim 4, wherein the instructions, when executed, further cause the computing system to:
generate a health-care avatar corresponding to an artificially intelligent healthcare provider, wherein the personalized experience interface includes the health-care avatar.

7. The at least one computer readable storage medium of claim 1, wherein the prescription information includes at least prescription identification information and dosing information.

8. A computing system comprising:
a processor; and
a memory having a set of instructions, which when executed by the processor, cause the computing system to:
select a given group of monitoring information to collect from a user, wherein the given group of monitoring information includes prescription information;
instruct a first computing device associated with the user to collect the given group of monitoring information;
determine that the given group of monitoring information is unavailable to be provided by the first computing device with the given group of monitoring information being used to identify at least one health condition of the user;
determine a class associated with the first computing device;
apply a machine learning model to the class associated with the first computing device to determine whether to instruct the first computing device to re-collect the given group of monitoring information;
instruct the first computing device to re-collect the given group of monitoring information based on the class associated with the first computing device and the machine learning model determining that the first computing device is to re-collect the given group of monitoring information;
obtain a batch of training data comprising a first set of a plurality of training computing device class features associated with re-reading requests;
process the first set of the plurality of training computing device class features by the machine learning model to generate an estimated need for a re-reading request;
compute a loss based on a deviation between the estimated need for the re-reading request and the re-reading requests associated with the first set of the plurality of training computing device class features;
update parameters of the machine learning model based on the computed loss;
in response to the given group of monitoring information being determined as being unavailable to be provided by the first computing device, generate an outreach event to request the given group of monitoring information from the user; and
in response to the given group of monitoring information being determined as being available, select an action pathway for the user based at least in part on the prescription information.

9. The computing system of claim 8, wherein the set of instructions, which when executed by the processor, cause the computing device to:
receive, responsive to a user fulfilling at least one prescription, a prescription notification indicating information corresponding to the at least one prescription and information corresponding to the user; and
identify, based on the prescription notification, at least one health condition of the user.

10. The computing system of claim 9, wherein the set of instructions, which when executed by the processor, cause the computing device to:
in response to the at least one health condition corresponding to at least one health condition of a plurality of predetermined health conditions, generate a message including instructions for downloading a user application; and
communicate, to a user account associated with the user, the message.

11. The computing system of claim 10, wherein the set of instructions, which when executed by the processor, cause the computing device to:
in response to an indication that the user initiated the user application, provide, at an application setup interface, a plurality of data gathering queries;
store user responses to the data gathering queries;
generate, using the user responses, a data structure corresponding to the user; and
generate, based on the data structure corresponding to the user, a personalized experience interface that includes a user avatar that models, based on at least the information corresponding to the user and the at least one prescription, a biological identity of the user.

12. The computing system of claim 11, wherein the set of instructions, which when executed by the processor, cause the computing device to:
provide, at a display of the first computing device associated with the user, the personalized experience interface.

13. The computing system of claim 11, wherein the set of instructions, which when executed by the processor, cause the computing device to:
generate a health-care avatar corresponding to an artificially intelligent healthcare provider, wherein the personalized experience interface includes the health-care avatar.

14. The computing system of claim 11, wherein the prescription information includes at least prescription identification information and dosing information.

15. A method comprising:
selecting a given group of monitoring information to collect from a user, wherein the given group of monitoring information includes prescription information;
instructing a first computing device associated with the user to collect the given group of monitoring information;
determining that the given group of monitoring information is unavailable to be provided by the first computing device with the given group of monitoring information being used to identify at least one health condition of the user;
determining a class associated with the first computing device;
applying a machine learning model to the class associated with the first computing device to determine whether to instruct the first computing device to re-collect the given group of monitoring information;
instructing the first computing device to re-collect the given group of monitoring information based on the class associated with the first computing device and the machine learning model determining that the first computing device is to re-collect the given group of monitoring information;
obtaining a batch of training data comprising a first set of a plurality of training computing device class features associated with re-reading requests;
processing the first set of the plurality of training computing device class features by the machine learning model to generate an estimated need for a re-reading request;
computing a loss based on a deviation between the estimated need for the re-reading request and the re-reading requests associated with the first set of the plurality of training computing device class features;
updating parameters of the machine learning model based on the computed loss;
in response to the given group of monitoring information being determined as being unavailable to be provided by the first computing device, generating an outreach event to request the given group of monitoring information from the user; and
in response to the given group of monitoring information being determined as being available, selecting an action pathway for the user based at least in part on the prescription information.

16. The method of claim 15, further comprising:
receiving, responsive to a user fulfilling at least one prescription, a prescription notification indicating information corresponding to the at least one prescription and information corresponding to the user; and
identifying, based on the prescription notification, at least one health condition of the user.

17. The method of claim 16, further comprising:
in response to the at least one health condition corresponding to at least one health condition of a plurality of predetermined health conditions, generating a message including instructions for downloading a user application; and
communicating, to a user account associated with the user, the message.

18. The method of claim 17, further comprising:
in response to an indication that the user initiated the user application, providing, at an application setup interface, a plurality of data gathering queries;
storing user responses to the data gathering queries;
generating, using the user responses, a data structure corresponding to the user; and
generating, based on the data structure corresponding to the user, a personalized experience interface that includes a user avatar that models, based on at least the information corresponding to the user and the at least one prescription, a biological identity of the user.

19. The method of claim 18, further comprising:
providing, at a display of the first computing device associated with the user, the personalized experience interface.

20. The method of claim 18, further comprising:
generating a health-care avatar corresponding to an artificially intelligent healthcare provider, wherein the personalized experience interface includes the health-care avatar, wherein the prescription information includes at least prescription identification information and dosing information.
